(19) United States
(12) Patent Application Publication
(10) Pub. No.: US 2003/0140924 A1
(43) Pub. Date: Jul. 31, 2003

(54) Therapeutic gas conserver and control

(76) Inventors: Alonzo C. Aylsworth, Wildwood, MO (US); Gregory R. Miller, Washington, MO (US)

Correspondence Address: Paul M. Denk, 763 South New Ballas Road, St. Louis, MO, 63141, US

(21) Appl. No.: 10/287,899

(22) Filed: Nov. 5, 2002

Related U.S. Application Data
(60) Provisional application No. 60/331,148, filed on Nov. 6, 2001.

(51) Int. Cl. A61M 16/00

(52) U.S. Cl. 128/204.26; 604/23; 128/204.23; 128/205.24

(57) ABSTRACT

A method and device for sensing inhalations and controlling the delivery of oxygen or other therapeutic gases to a patient. A flow-through sensor is adapted to provide a method and device to monitor various parameters of a patient's breathing and/or pulse oxygen saturation. Adjustments are made to the bolus delivery from a source of therapeutic gas to efficiently provide appropriate levels of therapeutic gas to the patient and to economically conserve therapeutic gas.

The present application claims priority from and hereby incorporates by reference the substance of Provisional Application No. 60/331,148, filed Nov. 6, 2001.

### BACKGROUND AND SUMMARY OF THE INVENTION

The present invention relates to an apparatus and method for the conservation and control of therapeutic oxygen when such oxygen is being delivered to a patient in ambulatory or non-ambulatory conditions. The present invention may also be applied to the conservation and control of other therapeutic gases to a patient.

It is common with home health care oxygen therapy for the patients to use an oxygen conserver, sometimes called a demand device or demand cannula. Use of conservers is currently for ambulatory purposes. It is common in the industry to use diaphragm-based sensors to detect patient inhalations. Upon detection of the patient's respiration, the sensor is used to trigger a bolus of oxygen to be inhaled by the patient. These sensors work quite well at short distances between a patient and the sensor, (provided the patient is not asleep or mouth breathing) usually up to distance of about 7 feet from the patient's nose to the sensor.

The present invention described and claimed herein describes the use of flow-through sensor technology. The present invention provides a device and a method for the efficient conservation of therapeutic oxygen and for the control of various oxygen deliveries, concentrating, and generating means when such oxygen is being delivered to a patient in ambulatory or non-ambulatory conditions. The present invention is capable of supplying therapeutic gas, including oxygen, to a patient during sleep (24 hours a day/7 days a week) as well as at rest or during exercise without the need for a device operator to make adjustments as a result of a change in the patient's activity level, respiratory rate, respiratory effort, pulse rate, and/or blood oxygen saturation level. The present invention monitors various parameters of a patient's breathing and/or pulse oxygen saturation and makes adjustments to the bolus delivery to efficiently provide appropriate levels of therapeutic gas to the patient and to economically conserve said therapeutic gas. In addition, the present invention can determine apnea events and may record the occurrence; provide a data log of apnea events; and provide notification or activate an alarm on the occasion of such events to the patient or a caregiver. The present invention can determine, assess, and adjust to the characteristics of a bolus delivery circuit, including the patient cannula, extension tubing, and any pressure or backpressure factors; all of which affect the bolus that a patient actually receives at their nose.

The present invention may allow the caregiver or operator to preset parameters for the bolus delivery amount and to set an allowable variance of the delivery parameters to define normal operating conditions. The present invention may include a Date/Time function for the determination of the most appropriate bolus delivery based on the time of day and to determine key parameters of respiration such as Breaths per Minute (BPM) and to factor the parameters into algorithmic determinations.

The present invention may be manually energized by a common power switch or through the use of a patient's inhalation as the energizing means. The present invention may be de-energized by use of a common power switch or by the absence of inhalations based upon a preprogrammed algorithmic determination.

Upon initial power-up and periodically thereafter, the present invention may use a self-calibration mode to ensure optimum sensitivity and capability versus environmental conditions and other factors such as component aging. The present invention may measure normal and rhythmic artifacts and use algorithmic means to adapt to such artifacts to minimize false detections and deliveries. A breath-to-breath time based lockout and a sensing hysteresis are important features that may be incorporated within the scope of the claimed present invention.

The present invention includes detection and memory means for event detection, diagnostic information, and the communication and display of such information and events. The present invention may use communications that will allow for external programming, the downloading of information, device configuration, and for the connection of external devices such as pulse oximetry devices and compliance monitoring. Pulse oximetry may be used as a feedback to control the bolus delivery. Such feedback may also be used for control during continuous flow delivery modes as well.

The present invention may be used for the efficient control of external oxygen delivery devices, concentrating devices, and/or generating devices. Various valve types and valve locations are taught which allow for bolus optimization and improvement of the bolus delivery in certain applications. In addition, the present invention may use various sensor locations and methods that enable other technologies to be developed. Additionally, the present invention claims the use of diaphragm based sensors to provide monitoring, control, and measurement capability.

In addition to the novel features and advantages mentioned above, other objects and advantages of the present invention will be readily apparent from the following descriptions of the drawings and preferred embodiments.

### BRIEF DESCRIPTION OF THE DRAWINGS

FIG. 1 is a representation of the Honeywell Honeywell Micro Switch AWM3100V Microbridge Mass Airflow/Amplified Sensor as used in a preferred embodiment of the present invention.

FIG. 2 is an illustration showing an application using the prior art Diaphragm Sensor.

FIG. 3 is an illustration showing an exemplary embodiment of the present invention.

FIG. 4 is an illustration showing an application using the prior art Diaphragm Sensor.

FIG. 5 is an illustration showing an application using the prior art Diaphragm Sensor.

FIG. 6 is an illustration showing an exemplary embodiment of the present invention.

FIG. 7 is an illustration showing an exemplary embodiment of the present invention.

FIG. 8 is an illustration showing an exemplary embodiment of the present invention.

FIG. 9 is an illustration showing the arrangement of devices for the performance of the Conserver Flow Test Method.

FIG. 10 is a representation of the Honeywell Honeywell Micro Switch AWM5000 Microbridge Mass Airflow/Amplified Sensor as used in the testing of the present invention.

FIG. 11 is a graphical representation of a test conducted to measure the performance of the present invention.

FIG. 12 is a graphical representation of a test conducted to measure the performance of the present invention.

FIG. 13 is a graphical representation of a test conducted to measure the performance of the present invention.

FIG. 14 is a graphical representation of a test conducted to measure the performance of the present invention.

FIG. 15 is a graphical representation of a test conducted to measure the performance of the present invention.

FIG. 16 is a graphical representation of a test conducted to measure the performance of the present invention.

FIG. 17 is a graphical representation of a test conducted to measure the performance of the present invention.

FIG. 18 is a graphical representation of a test conducted to measure the performance of the present invention.

FIG. 19 is a graphical representation of a test conducted to measure the performance of the present invention.

FIG. 20 is a graphical representation of a test conducted to measure the performance of the present invention.

FIG. 21 is a graphical representation of a test conducted to measure the performance of the present invention.

FIG. 22 is a graphical representation of a test conducted to measure the performance of the present invention.

FIG. 23 is a graphical representation of a test conducted to measure the performance of the present invention.

FIG. 24 is a graphical representation of a test conducted to measure the performance of the present invention.

FIG. 25 is a graphical representation of a test conducted to measure the performance of the present invention.

FIG. 26 is a graphical representation of a test conducted to measure the performance of the present invention.

FIG. 27 is a flow chart showing the Conserver Top Level Feature and Operational Diagram process and software of a preferred embodiment of the present invention.

FIG. 28 is a flow chart showing the Calibration and Configuration process and software of a preferred embodiment of the present invention.

FIG. 29 is a graphical representation of a test conducted to measure the performance of the present invention.

FIG. 30 is a graphical representation of a test conducted to measure the performance of the present invention.

FIG. 31 is a graphical representation of a test conducted to measure the performance of the present invention.

FIG. 32 is a graphical representation of a test conducted to measure the performance of the present invention.

FIG. 33 is a graphical representation of a test conducted to measure the performance of the present invention.

FIG. 34 is a graphical representation of a test conducted to measure the performance of the present invention.

FIG. 35 is a graphical representation of a test conducted to measure the performance of the present invention.

FIG. 36 is a flow chart showing the basic conserver embedded software and process of a preferred embodiment of the present invention.

FIG. 37 is a graphical representation of a test conducted to measure the performance of the present invention.

### DETAILED DESCRIPTION OF THE EXEMPLARY EMBODIMENT(S)

An apparatus and method for the conservation and control of therapeutic gas, such as oxygen.

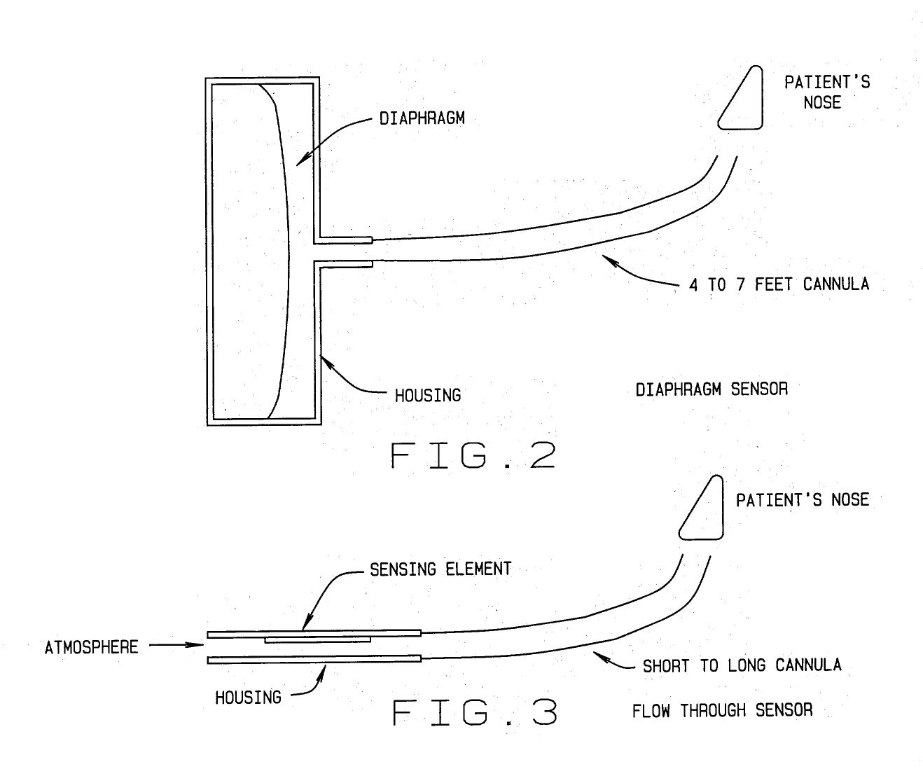

The prior art, as shown in FIG. 2, is a diaphragm-based sensor that uses a diaphragm enclosed in a housing. The diaphragm is enclosed in a housing that may or may not reference atmosphere. In either case, the patient must create sufficient vacuum, or inhalation strength, during an inhalation to pull the diaphragm and create an electrical or mechanical signal that will in turn provide delivery of a bolus of oxygen to the patient.

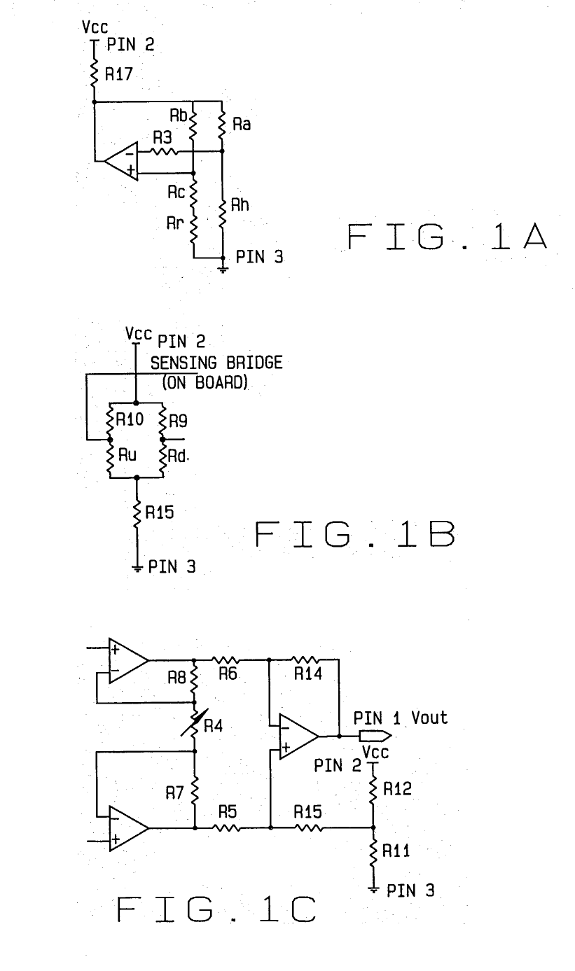

As shown in FIG. 3, an example embodiment of the present invention illustrates the application of flow-through sensor technology. The example embodiments of the present invention are described using the Honeywell Micro Switch AWM3100V Microbridge Mass Airflow/Amplified Sensor, FIG. 1. In the case of a flow-through sensor, such as Honeywell's AWM3100V, the patient can easily create sufficient and greater vacuum through the housing since there is very little backpressure. The inhalation is easily sensed, as even the smallest levels of airflow pass over the sensing element rather that having to pull a diaphragm surface forward for a response. The flow-through sensor that may be used in this embodiment of the present invention is sensitive enough to even detect minute mouth breathing through the nasal cannula. The low backpressure in the flow-through sensor design is superior in most applications of conserving devices.

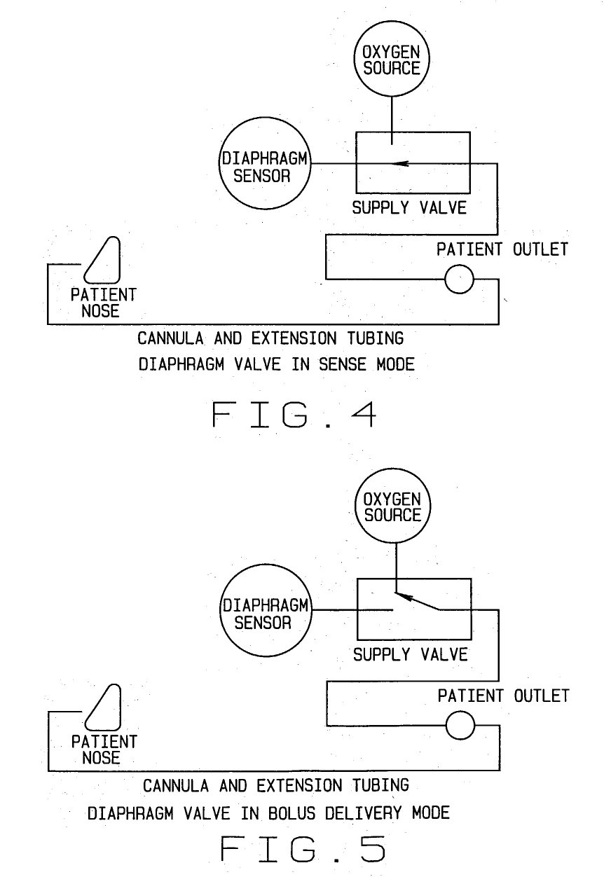

To deliver the bolus of oxygen to a patient, a valve means must be employed. The prior art illustrated in FIG. 4 shows a supply valve used to supply oxygen to a patient or to a diaphragm sensor. In Sense Mode, the inhalation of a patient draws a vacuum on the system starting from the patient's nose, through the cannula and extension tubing, through the patient outlet of the device, through the supply valve and to the diaphragm sensor. Upon the presence of a sufficient vacuum, the diaphragm will deflect and cause an electrical or mechanical response that will cause the supply valve to activate as shown in FIG. 5. When in Bolus Delivery Mode, the supply valve opens a connection to the oxygen source. Oxygen is allowed to flow from the oxygen source, through the supply valve, through the patient outlet, through the cannula and extension tubing to the patient's nose. When the Bolus Delivery Mode is complete, the supply valve closes relative to the oxygen source and reopens relative to the diaphragm sensor. At this point, the system is restored to Sense Mode as illustrated in FIG. 4.

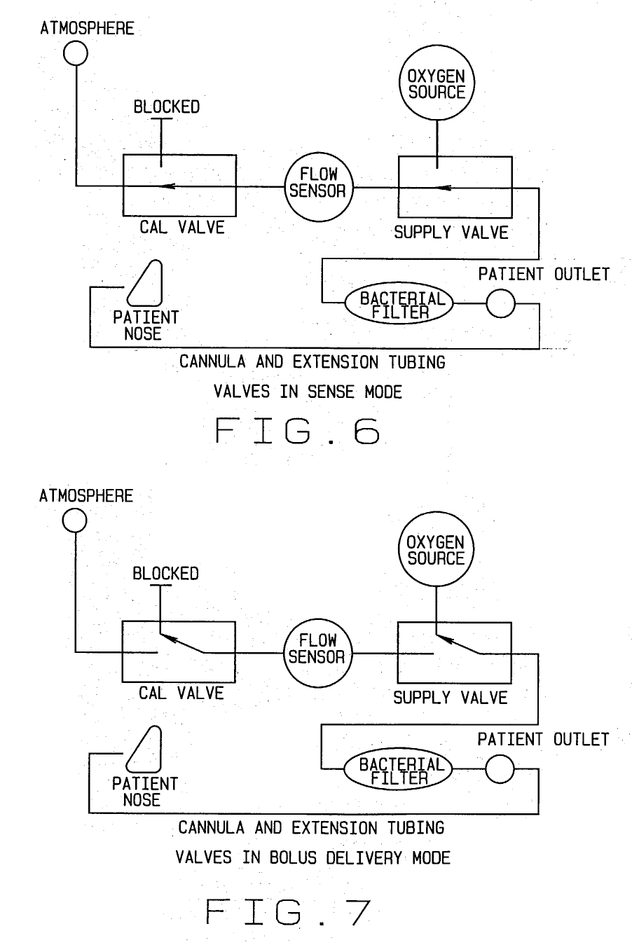

As shown in FIG. 6, an example embodiment of the present invention illustrates the application of flow-through sensor technology and an improved valve means. Similar to the prior art illustrated in FIG. 4, an oxygen source, a supply valve, a patient outlet, and a cannula and extension tubing are used. FIG. 6 illustrates an application using a flow-through sensor and a valve operating as a two-way pneumatic valve that for explanation purposes will be called, “cal valve”. The present example demonstrates the use of an atmospheric reference. As shown in FIG. 6, the supply valve connects the oxygen source and the sensing means to the patient's nose. The atmospheric side of the flow-through sensor is connected to the cal valve. The opposite side of the cal valve is open to the atmosphere to provide a reference to atmosphere for this example embodiment of the present invention. Typically, the atmospheric outlet to the cal valve is made through a length of tubing that is of an adequate length to prevent atmospheric air from entering beyond the cal valve during a patient's inhalation. Since the apparatus and the method of inhalation sensing described in the present invention is highly sensitive, it is possible to install a bacterial filter between the patient outlet and the supply valve without a substantial change in performance. Additionally, the present invention may include a bacterial filter anywhere in the patient circuit between the supply valve and the patient's nares. In Sense Mode as shown in FIG. 6, the patient's inhalation creates a vacuum that pulls atmospheric air into the tube between the atmosphere and the cal valve. Gaseous airflow is allowed to flow through the flow sensor and along the sensing element located inside of the flow sensing element, through the supply valve, through the bacterial filter, through the patient outlet, through the cannula and extension tubing, and info the patient's nose. The airflow over the sensor's sensing element will create a response causing the valves to shift to the Bolus Delivery Mode as shown in FIG. 7.

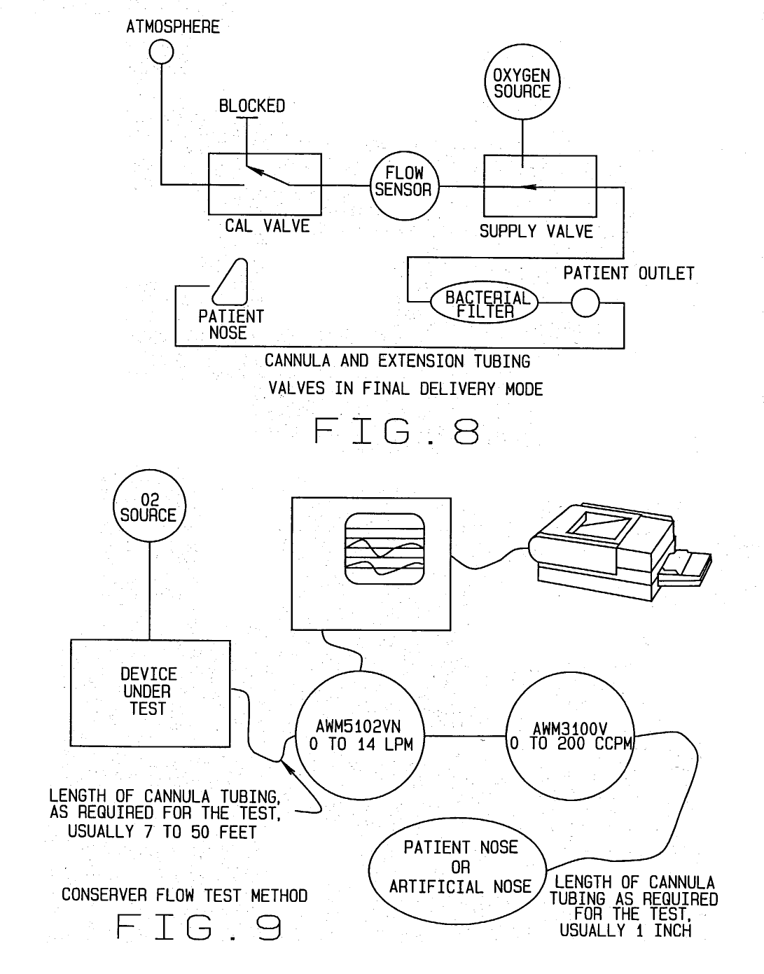

In the Bolus Delivery Mode, FIG. 7, the cal valve shifts to the blocked, or no-flow, position and the supply valve shifts to open relative to the oxygen source. This causes a bolus of oxygen to be delivered to the patient. Upon completion of the Bolus Delivery Mode, the supply valve shifts back to a closed position relative to the oxygen source and opens to flow relative to the flow-through sensor as shown in FIG. 8. In the Final Delivery Mode as shown in FIG. 8, the cal valve remains in the blocked position. When the bolus is delivered, pressure builds in the system between the patient's nose and the supply valve. This pressure build up consists of valuable oxygen that is intended for use by the patient. If the cal valve were not present to block the atmospheric side of the flow-through sensor, then much of the pressure would be relieved through the atmospheric side of the flow sensor thus causing a waste of therapeutic gas, or oxygen in this case. Therefore, the cal valve remains in the blocked position for an amount of time to allow the complete delivery of the oxygen to the patient. Upon the completion of the Final Delivery Mode the system returns to the Sense Mode as shown in FIG. 6.

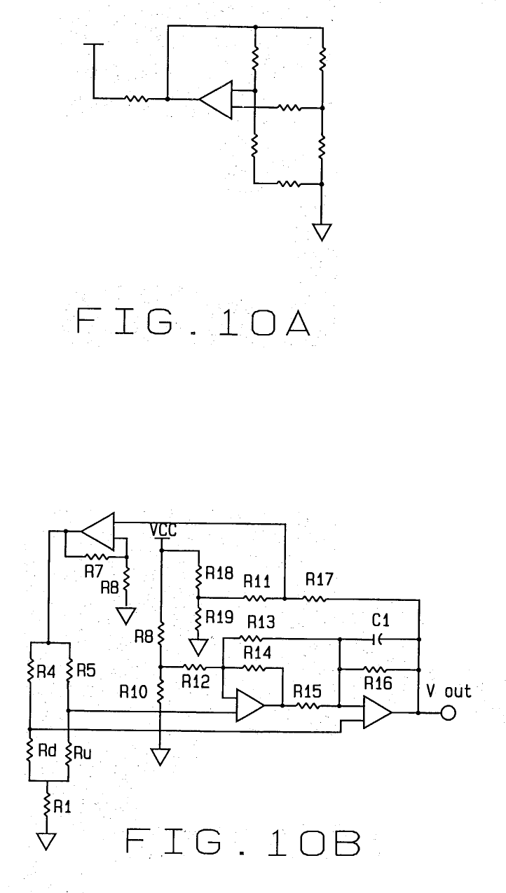

A Conserver Flow Test Method, as illustrated in FIG. 9, was developed to demonstrate the differences in performance between the prior art diaphragm-type systems and the flow-through sensor as disclosed in the present application. An oxygen source is connected to supply gas to the device under test. A length of cannula and extension tubing exits the device under test. Different applications of the present invention will require longer tubing lengths, thus giving a patient greater mobility, range, and distance from their supplemental oxygen. As a result, multiple cannula and extension tubing lengths that exit the device under test were utilized. As shown in FIG. 9, a length of cannula enters a Honeywell Micro Switch AWM5102VN High Flow Mass Airflow/Amplified sensor, FIG. 10, rated for to measure airflow from 0 to 15 liters per minute. This sensor allowed measurement and recording of the actual bolus being delivered. The AWM5102VN is connected by a short piece of tubing to an AWM3100V Microbridge Mass Airflow/Amplified Sensor. The AWM3100V sensor is much more sensitive as it is rated for measurements ranging from 0 to 200 ccpm, which is within a suitable range to allow measurement of the inhalations and exhalations from a patient or artificial nose. The AWM3100V is connected to a nose by a short piece of tubing, about one-inch in length. The testing of the present invention involved the manufacture of an artificial nose capable of creating repeatable inhalations of varying degrees to create standard test conditions. The two airflow sensors are connected to a digital storage scope and test plots are printed to paper for further analysis and for historical purposes. Our prototype device embodiment of the present invention was called the FLT. Our prototype uses a Parallax BS-2 Basic Stamp microprocessor. Because this processor uses a BASIC language compiler the processing speed will be about 20% slower in response versus a production model that would use an assembly language based processor. Some applications of an assembly-based processor would be hundreds times faster. A series of comparative tests were performed of the FLT and a diaphragm-based current device, herein called the OM-400.

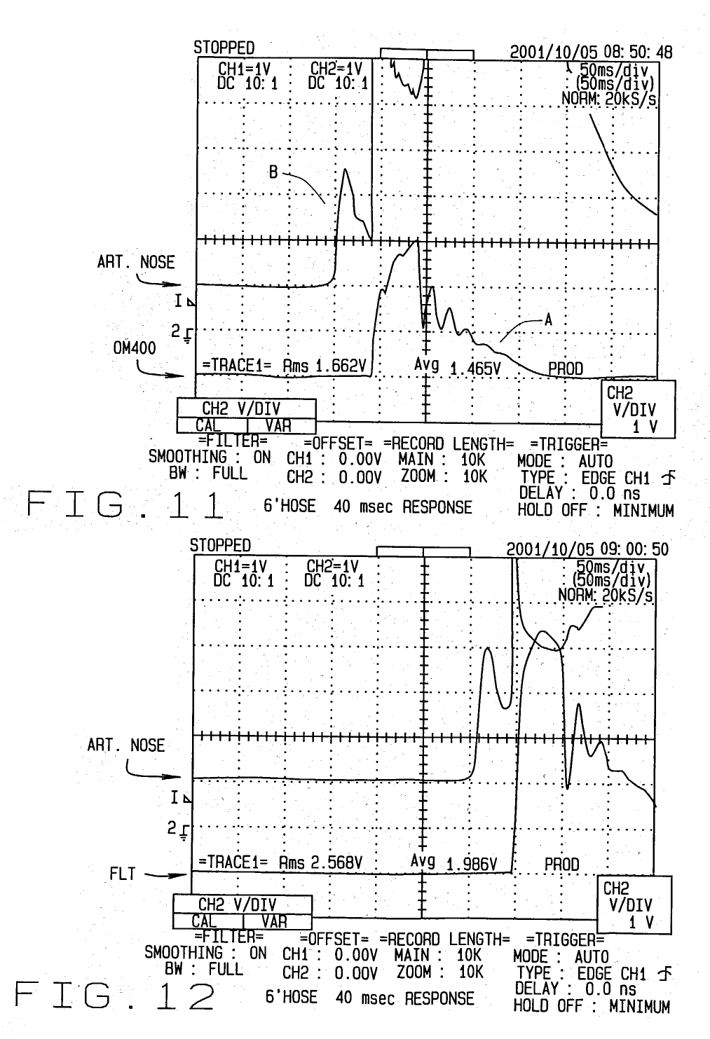

FIG. 11 is a printed copy of a response time of the OM-400 versus the artificial nose, whereas the OM-400 is the Device Under Test. The bottom trace, A, is the AWM5102VN sensor and represents the response time of the OM-400. The top trace, B, is the output of the AWM3100V sensor. The top trace, B, shows a measure of the initial inhalation and then displays the bolus being delivered. The test record as shown on FIG. 11, demonstrates the artificial nose making a rapid inhalation, shown at trace B. Approximately 40 msec later the bolus is delivered. For this test, the cannula length is 6 feet long.

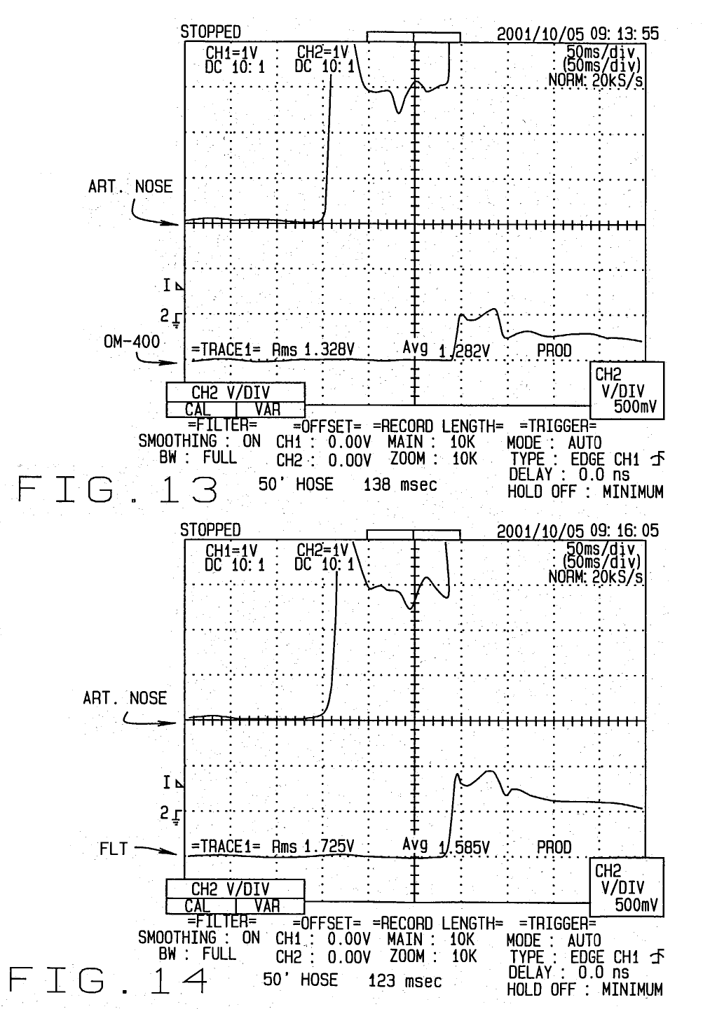

FIG. 12 is the same test performed in FIG. 11 except that the FLT device is the Device Under Test. Note that the rapid inhalation causes a response about equal to the OM-400 of about 40 msec. The bolus delivery is greater because the valve of this embodiment of the present invention is less restrictive to flow. The lower restriction does have a minor effect on the test data but as may be seen below, this difference is inconsequential.

FIG. 13 illustrates the same test performed in FIG. 11, except the 6 feet of cannula tubing is replaced with a 50-foot section. Note that once again there is a rapid inhalation from the artificial nose. This inhalation produces a bolus delivery about 138 msec later. The response takes more time because of the longer tubing.

FIG. 14 illustrates a similar test using the FLT device, this time with 50 feet of tubing. The response is almost equal to the OM-400 results, except the device could be about 20% faster, or more, if the assembly language based device previously discussed were used.

Another response test was performed and illustrated in FIG. 15. In this test, 6 feet of tubing is reinstalled in the test setup. The OM-400 is the Device Under Test. Previous tests used a rapid inhalation. This test uses a more normal, patient-like, inspiration as can be seen in trace B. The resulting inhalation causes a response at 1.75 volts.

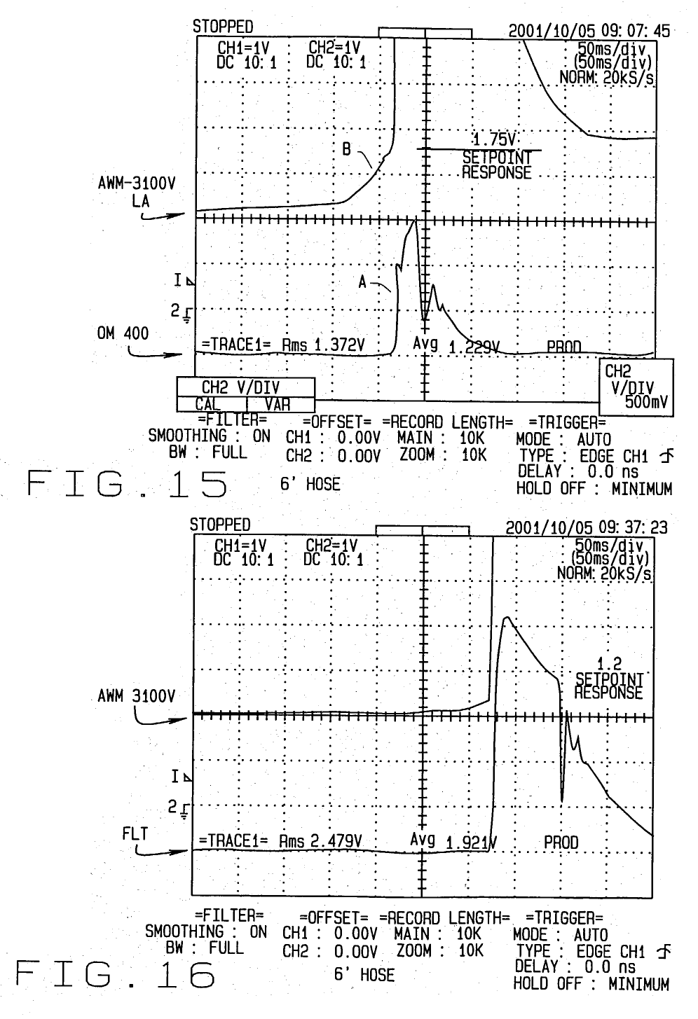

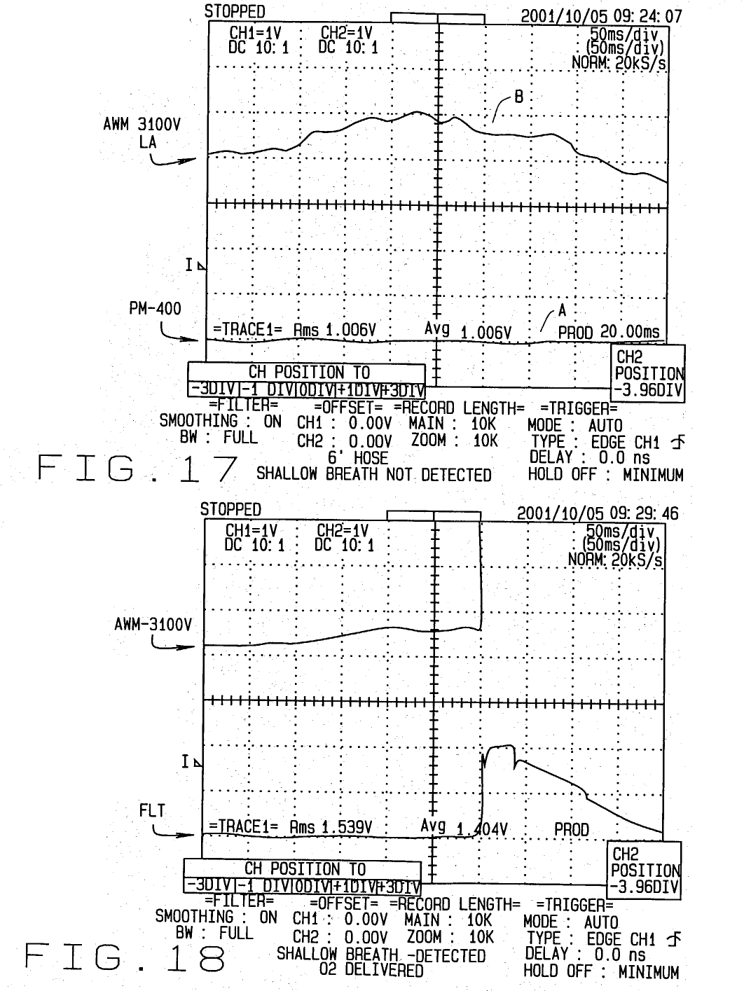

The same test was performed and illustrated on FIG. 16 except the FLT device is the Device Under Test. Six feet of hose was used in this test for the measurement of the response time. The test indicates a voltage response at 1.2 volts and much earlier in the inhalation.

Manufacturers strive to deliver the bolus as early as possible in the inhalation (to minimize respiratory dead space) because the earlier the response during an inhalation, the less oxygen required in the bolus delivery to achieve the desired therapeutic result. A number of advantages arise as a result of early delivery and the increased capability of gas conservation.

Sometimes patient's inhalations are very shallow. A shallow breath is demonstrated in FIG. 17. In this test the OM-400 is the Device Under Test. In this test, a very shallow inhalation at trace B does not cause a response from the OM-400 on trace A. The level of the inhalation is too low for the OM-400 to respond to. As a result the bolus of oxygen is not delivered.

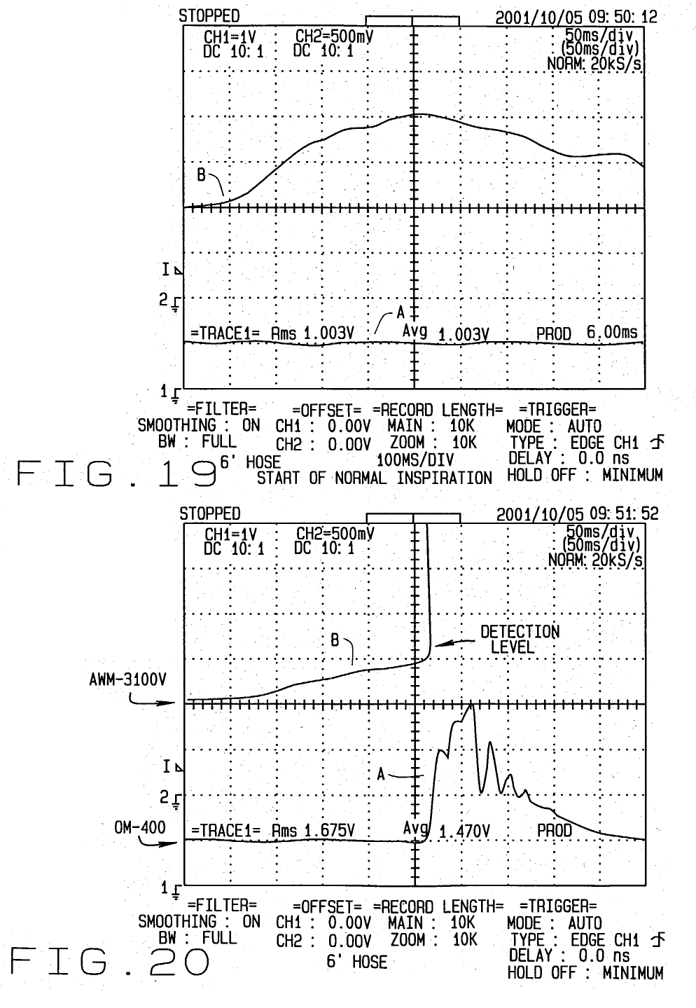

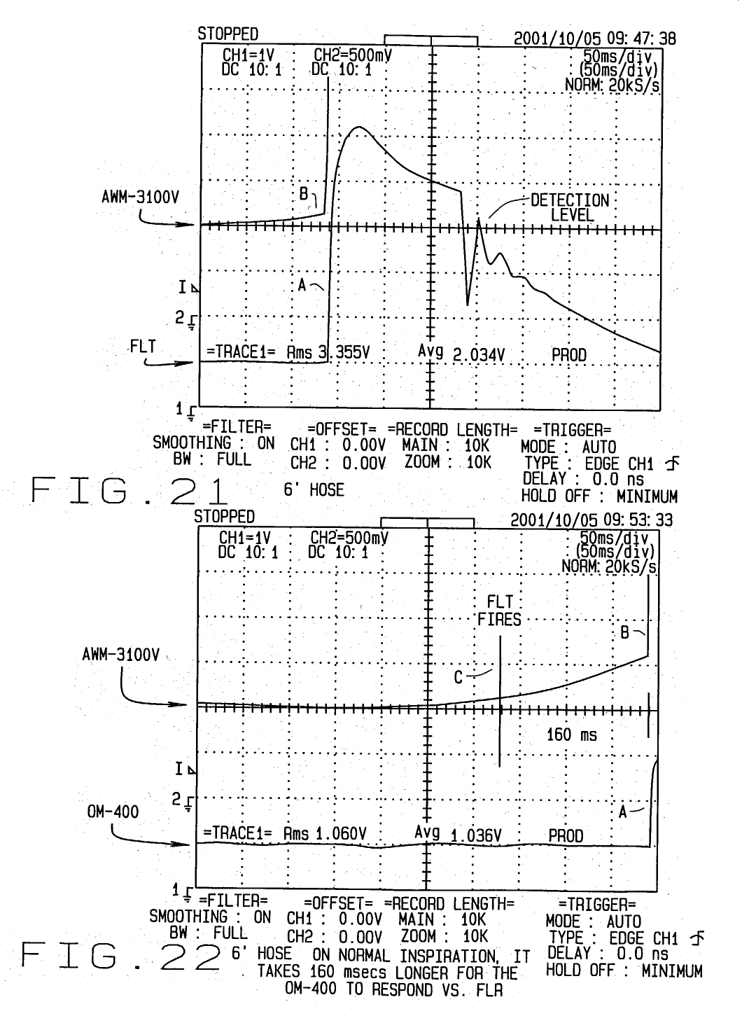

The same test was performed in FIG. 18, except the FLT device is the Device Under Test and the inhalation is even shallower and thus more difficult to detect. The test shows the FLT device was able to deliver the bolus to the patient. FIG. 19 illustrates the start of a normal inhalation at trace B. No device is connected at A, note that 6 feet of hose is used in this test setup and that the scale on the graph is set for 100 msec. Referring to FIG. 20, this test uses the same type of normal inhalation as displayed in FIG. 19. Also note that the scale is set to 50 msec per division in order to zoom in on the inhalation measurement representation. The inhalation at B causes a bolus delivery at A. FIG. 21 shows the same test except the FLT device is the Device Under Test. The inhalation at B causes a Bolus delivery at A. FIG. 22 displays the results from the same test as in FIG. 20. An inhalation at B causes a bolus delivery at A. The results from the test illustrated in FIG. 21 are drawn in at C. This test, FIG. 22, indicates a response by the FLT device of about 160 msec earlier than the OM-400 device.

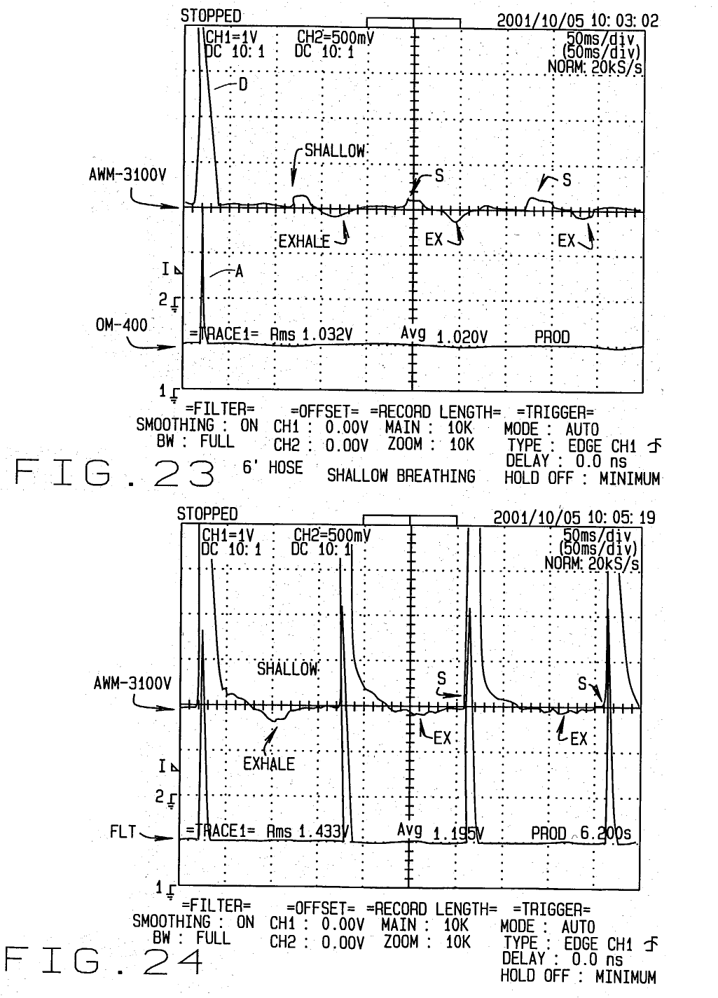

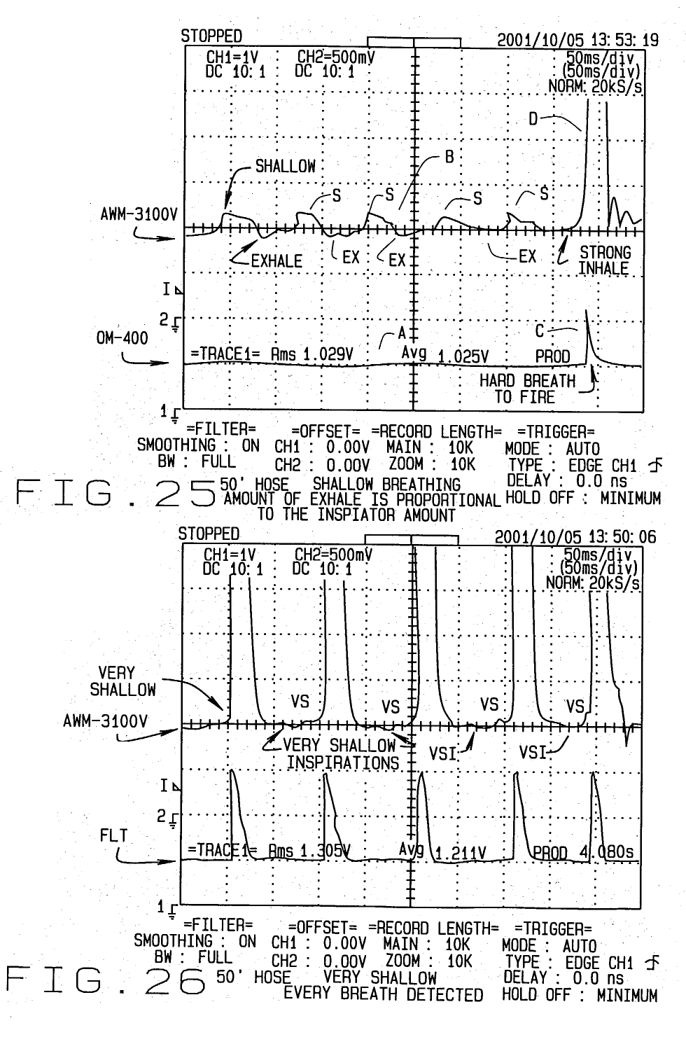

FIG. 23 is a test having the OM-400 as the Device Under Test and using 6 feet of cannula hose and one strong inhalation followed by a series of shallow breathing. The scale is set to 2 seconds per division in order to get more breaths onto one graph page. The initial strong inhalation at D causes a bolus delivery at A. The next series of shallow breathing does not result in a bolus delivery by the OM-400. This test illustrates the occurrence wherein patients with some disease states breathe very shallow during rest or sleep.

The same test was performed in FIG. 24 except the FLT device is the Device Under Test and the inhalation is even shallower and thus more difficult to detect. In FIG. 24, the FLT device detects every inhalation and a bolus is delivered upon each inhalation.

An additional test was performed, FIG. 25, using the OM-400 as the Device Under Test, 50 feet of hose, and a series of shallow breathing. There were no detections or bolus deliveries for any of the shallow inhalations at traces B and A. The strong inhalation at D produced a small Bolus delivery at C. This strong level of inhalation is not normal for a patient in a disease state and is shown only to prove the OM-400 is operating.

The same test was performed in FIG. 26 except the FLT device is the Device Under Test and the inhalation is even shallower and thus more difficult to detect. In FIG. 26, the FLT device detects every inhalation and a bolus is delivered upon each inhalation.

The above information demonstrates and teaches the differences in diaphragm and flow-through sensing and valve means, and the inherent advantages of the flow-through sensor technology. Additionally, with the knowledge contained in the aforementioned discussion, it is possible to discuss some of the applications and inventions that are enabled by the disclosed technology.

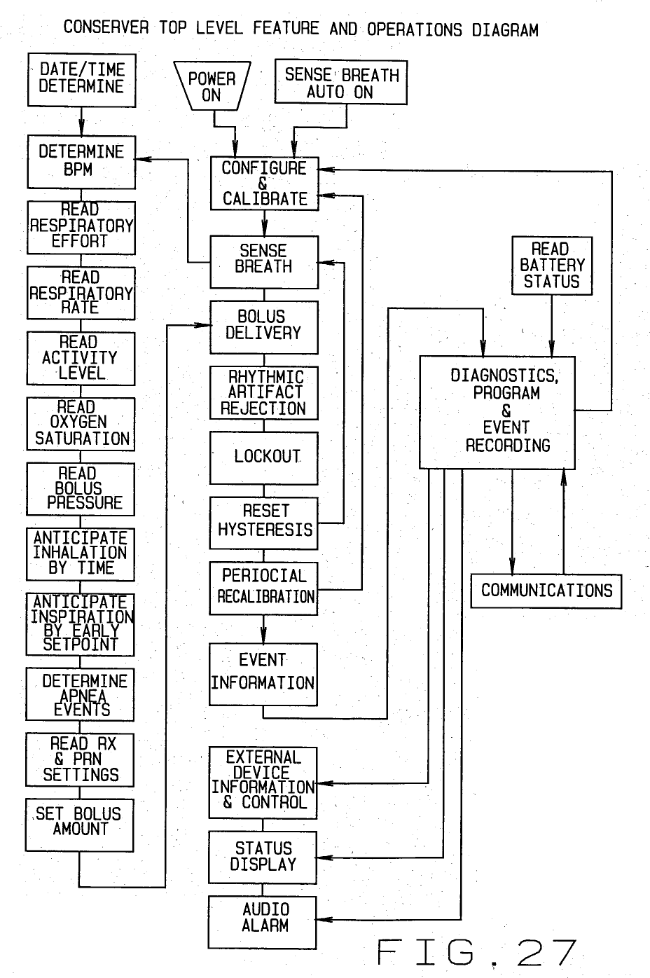

FIG. 27, Conserver Top Level Features and Operational Diagram, will be used as a guide for the following discussion. The items in FIG. 27 are demonstrative of example embodiments described and claimed in the present application.

The first block is the Power On block. This represents a normal on or on/off switching means for a device. It is intended that if an “on” only button were used, then this device would incorporate an “auto-off” feature.

The next section is the Sense Breath Auto ON block. This block represents the ability to have a sensor means to monitor the patient cannula and to automatically turn the device on during the first inhalation received. An example of a mechanical switch having the sensitivity to activate a conserver device when triggered by a patient's inhalation is manufactured by MPL. A conserver having such a switch would be simple to use for a patient. An auto-off feature could also be added. The Configure & Calibrate block represents the ability of the present invention to automatically calibrate the flow-through sensor and circuitry. Environmental, electrical, mechanical, and aging effects change the performance characteristics of any sensor. Through algorithmic means, the present invention seeks to optimize its performance level.

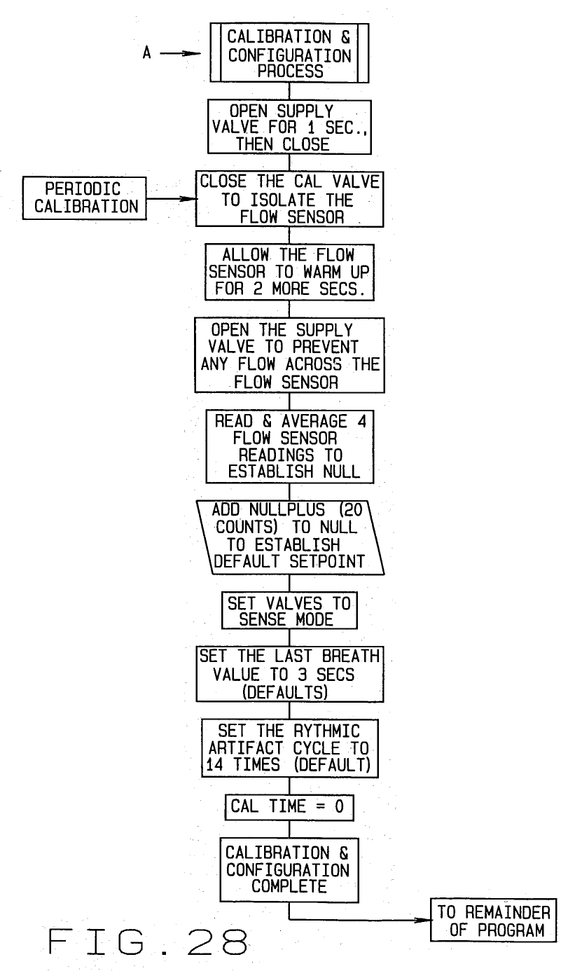

An example of the Calibration and Configuration Process algorithm is shown at FIG. 28. The supply valve opens to therapeutic gas flow for one second to flush out the cannula hose and the flow sensor, and then closes. The cal valve then closes to flow to isolate the flow-through sensor from atmosphere. The flow sensor is given another two seconds to warm up. This no-flow situation allows the electronics to establish a no-flow null level and to work off of this level for completing algorithmic expressions. The null level is read and averaged to establish the variable “Null”. In the preferred method of operation, a microprocessor with a 10 bit A/D converter is used and a setpoint level at Null+20 counts for the bolus to be delivered is applied.

When using the present invention in a setpoint mode as described herein, the bolus will be delivered whenever the enabling inhalation is performed and rises above the Null setpoint level. There are other methods of determining the setpoint that will trigger the delivery of a bolus by the FLT device. To complete the calibration sequence the valves are set to the Sense Mode as shown in FIG. 6. The invention configures itself at this point by setting variables to the desired state or value for the type of desired operation or control required. Configuration can be performed in hard written code, through user means, or through electronic communication means.

After the calibration and configuration are completed, the process of sensing inhalations begins as indicated at the Sense Breath block of the algorithm shown on FIG. 28. In this example, inhalations are sensed at 20 counts above Null. Some of the configuration variables determine the factors to be considered during the first few bolus deliveries. After the first few deliveries, the algorithmic means adapts to the patients breathing.

The process proceeds to the Date/Time Determination and Determine BPM, or Breaths Per Minute blocks. The microprocessor reads the time between breaths and calculates the BPM with the aid of the Date/Time Clock. This may be accomplished through other timing means, however. The BPM may be displayed, stored in memory, and/or remotely communicated to external devices including through telemetry, to control external devices, and used for algorithmic means as will be further described herein.

Readings of Respiratory Effort, Respiratory Rate and Activity Level are usually inter-related. Today, in home care situations a patient on a continuous flow of oxygen or pulsed through a conserver may be relaxing on a chair, and may then desire to get up and perform a minor task. According to the prior art, the activity that the patient is now performing requires the patient to go to the source of oxygen and increase the flow of oxygen to a higher level to compensate for their increased activity. This effort alone could tax the patient. The present invention includes the ability to sense this activity through the nasal cannula. The sensor used to detect inhalations can also profile the artifact created and determine that activity has been intitiated. This allows the flow of oxygen to be increased to a new preset level. This activity will cause an increase in respiratory effort and respiratory rate. These two factors, along with the activity level, may also be considered in the algorithm for determining the amount of increase in the bolus, if any, that will be required. The caregiver, the device operator, the patient or others may preset limits on the amount of increase as desired. Preset limits may include the method of setting upper and lower limits as fixed amounts, as variable amounts, as upper and lower limits as fixed amounts with varying amounts between the fixed amounts, or as variable amounts as the upper and lower limits and varying amounts between the varying outside limits.

The algorithm may react to a decrease in activity, respiratory effort, and/or respiratory rate, to initiate a corresponding decrease in the delivery or settings. For example during REM sleep, there is a marked decrease in minute volume and/or irregular breathing patterns and a corresponding decrease in blood oxygen level, therefore the process may be reversed. In REM sleep it may also be desired to increase the bolus size as determined by minute volume. The process is adapatable to provide an increase in bolus delivery when it may be required in lieu of a decrease, and conversely when a decrease in bolus delivery may be required in lieu of an increase.

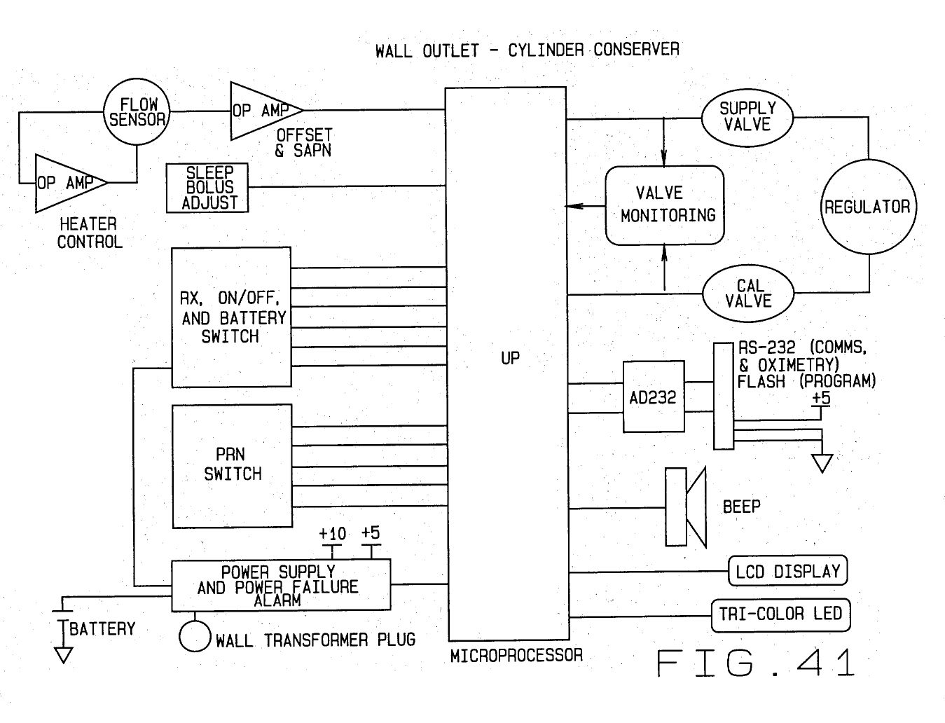

A continuous flow may also be automatically energized or de-energized based on the activity, respiratory effort, minute volume, or respiratory rate. This feature may be manually or automatically selected. Since an example embodiment of the present invention allows for profiling sleep waveforms, it is possible within the scope of the invention to affect changes in delivery as a result of sleep and and/or irregular breathing patterns. Date/Time may also be algorithmically factored into these items. Information on activity, respiratory effort, and/or respiratory rate may be displayed, stored in memory, and/or remotely communicated to external devices including through telemetry and used for algorithmic means and to control external devices. A waveform displaying activity detected by the flow-through sensor is displayed at FIG. 35.

The Read Oxygen Saturation block operates in many ways like the aforementioned respiratory effort, rate, and activity level readings. At this point, the actual patient oxygen saturation is monitored and controlled using pulse oximetry that is common in the art. The patient's oxygen saturation in their blood and/or heart rate are measured and a corresponding change in the delivery of oxygen or therapeutic gas, such as initiating continuous flow or conserving bolus deliveries, is made in response to any changes in the patient's oxygen saturation level. Increases or decreases in heart rate (tachycardia/bradycardia) and/or arrhythmia's are measured and a corresponding change in the delivery of oxygen or therapeutic gas may be made. For example, an adult's heart rate will typically increase in response to low oxygen saturation and a neonate heart rate drops with a drop in saturation. Monitoring may be continuous or sampled to reduce artifact. Pulse oximetry capability may be internally or externally installed to the device. Pulse oximetry information may be displayed, stored in memory, remotely communicated to external devices such as through telemetry, used for algorithmic means, and to control external devices.

The pulse oximetry information may be algorithmically compared to any or all of the following: activity, respiratory effort, respiratory rate, date and time, bolus pressure, and other factors either in real time and/or with data stored in memory to control the Bolus Delivery or continuous mode and the bolus amount or flow rate delivered therein. Additionally, pulse oximetry functions as a means for artifact analysis and identification of malfunctions. It is common, for example, for a patient attached pulse oximetry probe to fall off at night or become dislodged. The present invention uses the aforementioned means to then compare data to validate new data, to issue an alarm, to record and/or manipulate the data in memory, to act on the results, and to use such data to control external devices such as through telemetry means.

The Read Bolus Pressure block is provides for the determination of the characteristics of the pneumatic delivery system connected to a delivery device. When a bolus is delivered, the back pressure created in the delivery system can be measured and interpreted to suggest changes in the delivery time, amounts, and any other factor that may be required depending on the application. In an example embodiment of the present invention, a pressure transducer may be placed between the Supply Valve and the Patient Outlet as were shown on FIG. 6.

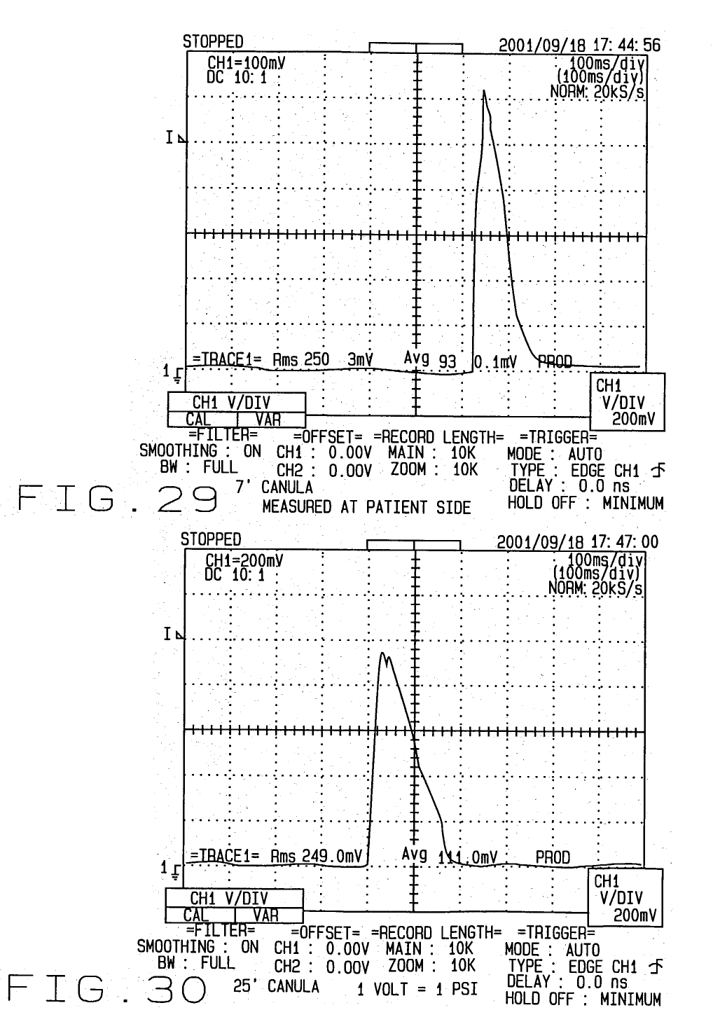

During the testing of the FLT as discussed above, when a bolus was delivered a pressure waveform was recorded on the digital storage O-Scope. The first test conducted measured the pressure waveform with a 7-foot patient cannula attached to the patient outlet. The resulting waveform is shown on FIG. 29. The bolus of oxygen is quickly delivered as illustrated with the pressure waveform. The same test was then performed except with 25 feet of patient cannula attached to the patient outlet. The test results are displayed on FIG. 30. Notice that the bolus of oxygen was not delivered as quickly. The test was performed again, now with 50 feet of cannula this time. The results are displayed on FIG. 31. Notice that the delivery takes even longer because of the increased backpressure resulting from the longer cannula tubing. This clearly illustrates the effects of patient circuit backpressure on the shape of the bolus. It may be desired in some applications to adjust the bolus delivery to match the backpressure created in the patient circuit. Using a microprocessor and associated algorithmic means, it is possible to shape the bolus to defined levels and results. Additionally, it may be desired to use a proportional valve or orifice means. This would allow the microprocessor to select an orifice size to match the desired delivered bolus. Various fixed orifice devices may also be selected. Also, it may be desired to simply add a switching means to allow the device operator to select the desired bolus manually. For example, the operator may desire to change from a 7-foot cannula to a 50-foot cannula and simply selects from 7 feet to 50 feet on a manual selector switch. One reason this is important was partially illustrated on FIG. 29. Note how strong the delivery occurred. This situation can create an uncomfortable POP in the patient's nares. Thus, it may be desirable to select a valve means or orifice means that is a compromise between patient comfort and speed of delivery.

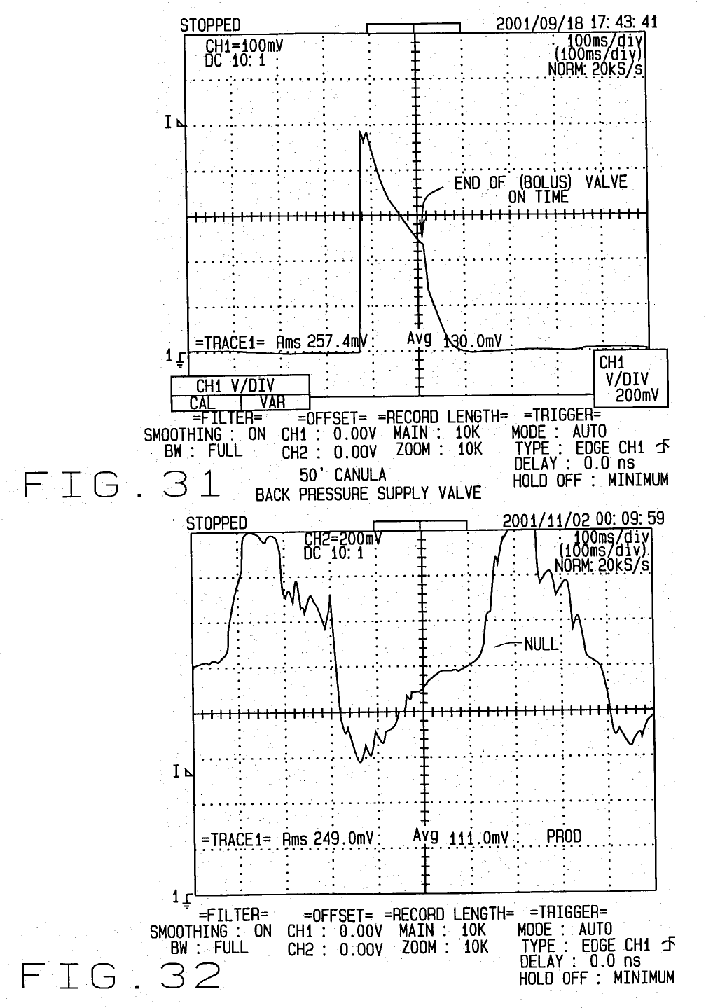

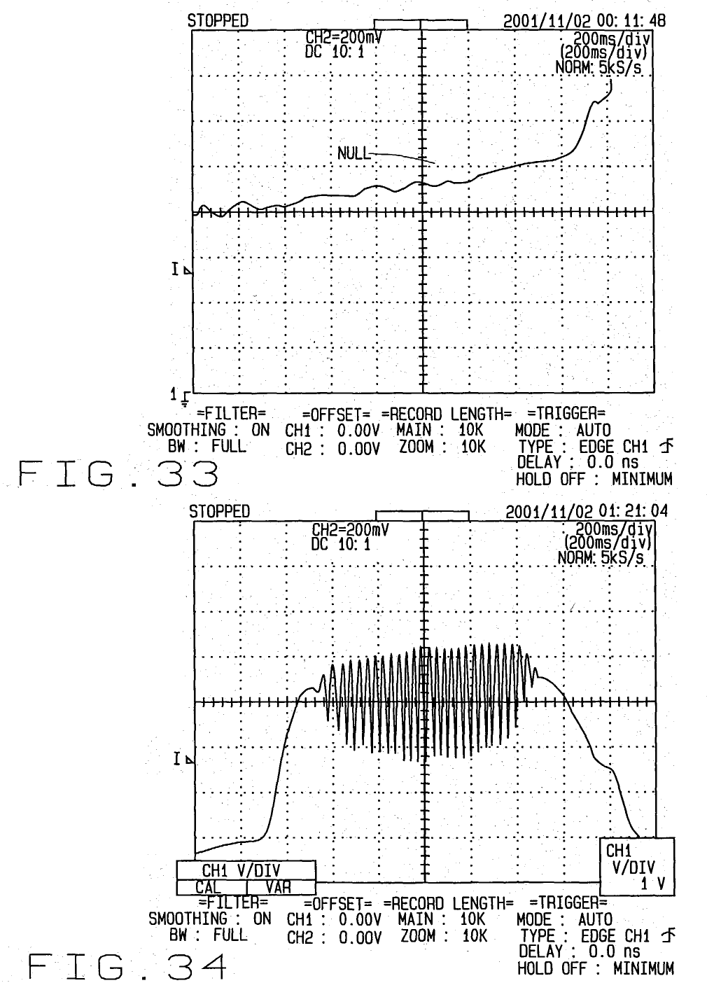

The Anticipate Inhalation by Time and Anticipate Inhalation by Early Setpoint blocks are steps taken to anticipate the next breath that a patient will be taking. By anticipating an inhalation, it is possible to deliver a bolus just prior to an inhalation. This method greatly reduces the amount of oxygen that needs to be delivered to a patient to achieve equivalent therapeutic results and thus increases the range of an ambulatory patient with a fixed supply of oxygen. Additionally, this enables the use of other technologies such as reduced size and reduced energy oxygen concentrators to be used with the present invention. When a patient is performing activity at a constant level or is at rest their breath rate is relatively constant, this is called rhythmic breathing. Under these conditions, it is possible to use a timing function to establish the breath rate and the time of the next inspiration. It is possible to anticipate the next inhalation by time. It is then possible, to deliver the bolus early, for example 100 msec earlier, than the onset of an inhalation. A dual lumen cannula may be used to monitor the inhalations with a flow sensor, or the single lumen flow sensor may be used. It may be necessary to occasionally revert back to the normal delivery mode in order to reestablish a baseline for the onset of the next inhalation. The bolus amount may be a specific amount for the normal delivery mode and a smaller amount for the anticipated delivery mode. To anticipate an inhalation by early setpoint level, monitor the flow waveform shown in FIG. 32. FIG. 32 displays the waveform of two inhalations about the null point at a scale of 1 sec per division. FIG. 33 also displays the onset of an inhalation but at a scale of 200 msec per division, thus allowing greater detail for analysis. As described above, an earlier example had Null Setpoint Level 20 counts above Null. This means that the FLT device will start the delivery of oxygen above the Null Setpoint Level. Under the conditions of rhythmic breathing, the next inhalation can be anticipated by lowering the Null Setpoint Level to a level less than Null, for example, Null—200 counts, so as to always consistently deliver the Bolus prior to the actual inhalation and thus enabling the same aforementioned advantages, including a substantial decrease in the amount of therapeutic gas that is required by a patient.

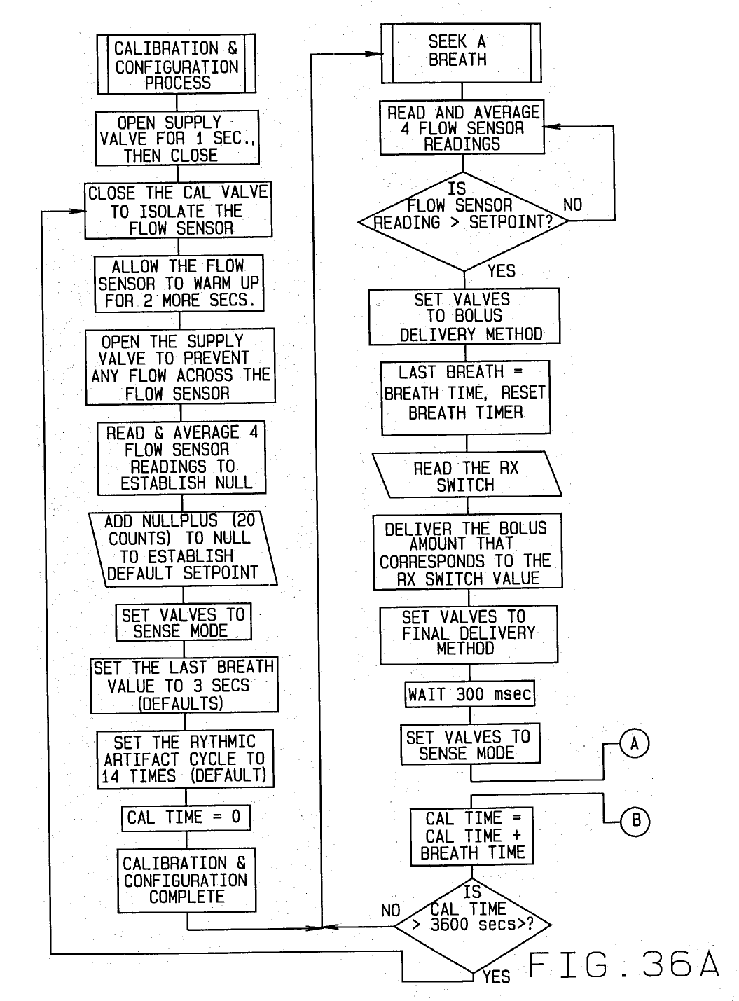

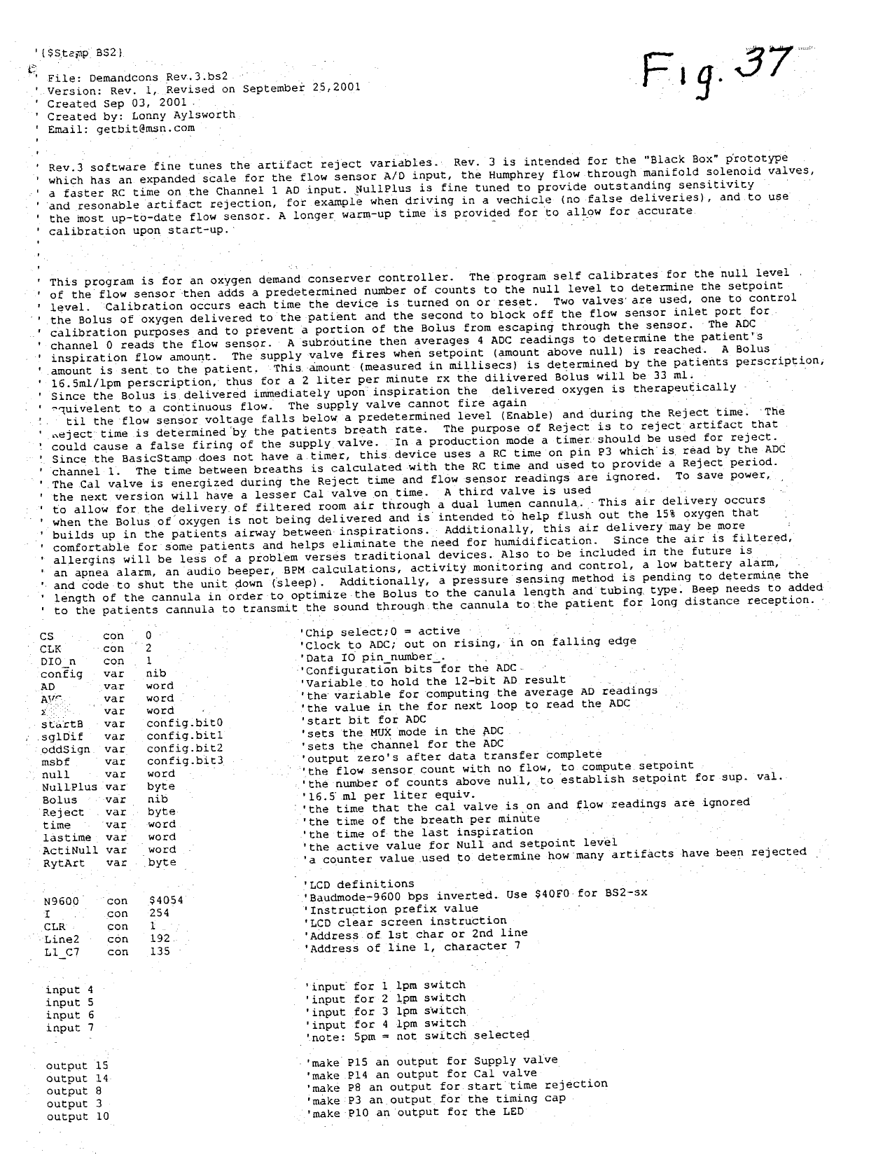

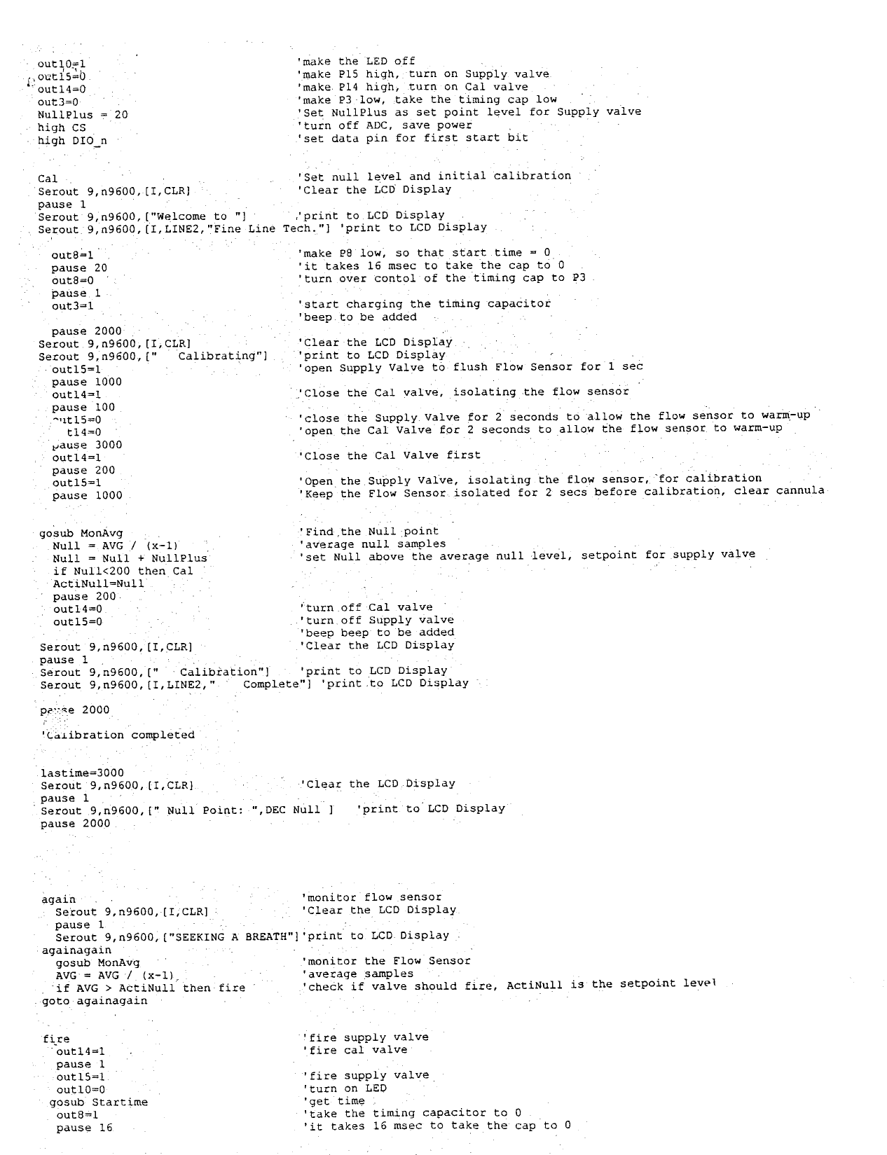

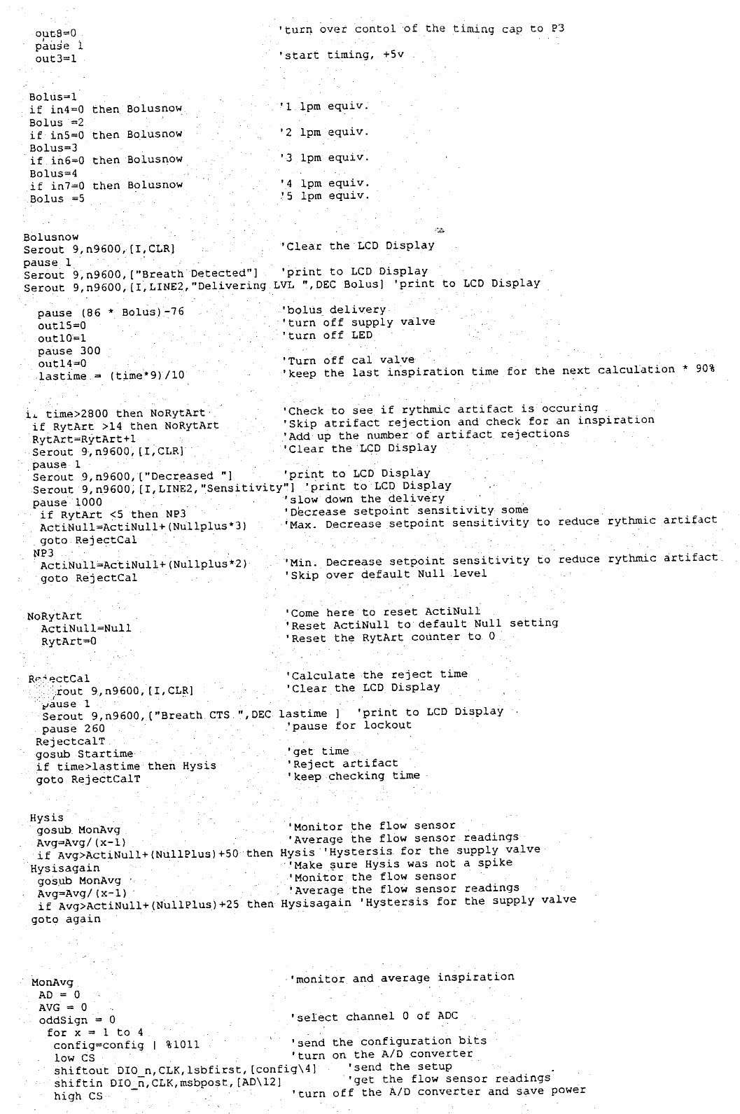

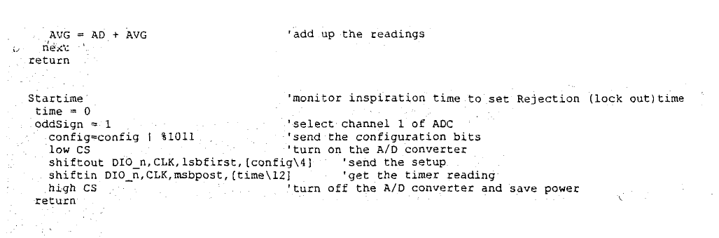

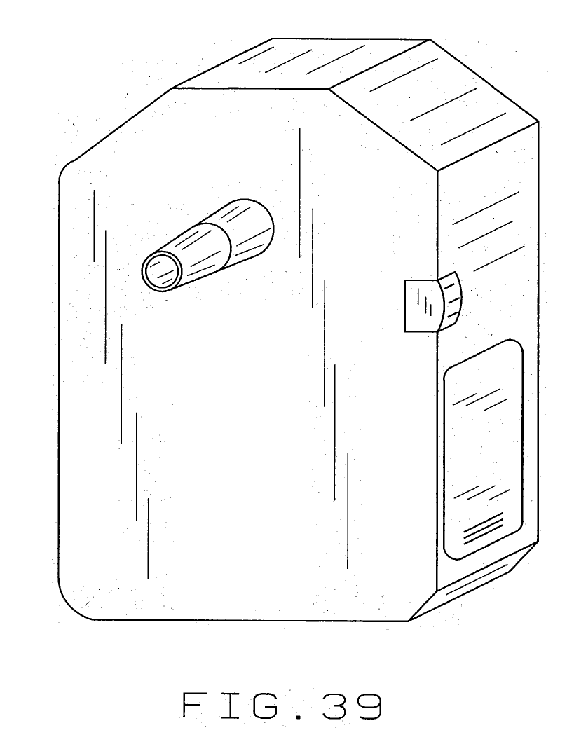

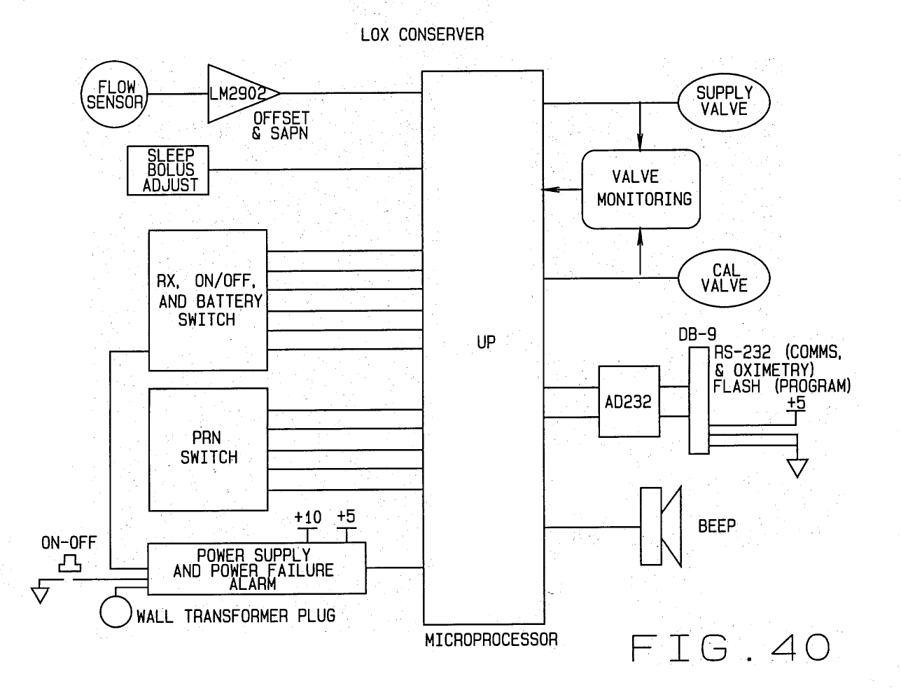

The Determine Apnea Events block, also covering hypopnea and snoring events, is a means to monitor and report apnea events within the scope of the present invention. Apnea is well known in the art of respiratory care. The present invention monitors inhalations for apnea events and may selectively alarm the device operator when such an event or series of events occurs. Information on apnea events may be displayed, stored in memory, used for algorithmic determinations, or remotely communicated for storage or control of external devices. Such information will contain data such as, but not limited to, the number of apneas, the longest apnea event, the apnea index, and the average length of an apnea event. All data is referenced to the Date/Time clock. Hypopneas and snoring activity may also be monitored and likewise recorded when a dual lumen cannula is used. Information on Hypopneas and snoring, including Hypopnea index and snoring index (a ratio comparing Inspiratory Snore data to breath rate) may be displayed, stored in memory, used for algorithmic determinations, or remotely communicated for storage or control of external devices. An inhalation waveform containing snoring is displayed on FIG. 34.

The Read Rx and PRN Settings block provides for multiple liter flow levels and limits as previously discussed in the Activity Level, Respiratory Effort, and Respiratory Rate block discussed above. The microprocessor or other electrical or mechanical means reads or otherwise corresponds to mechanical switch inputs, algorithmic means or communication means including external communication means, including telemetry to achieve the desired results as discussed above. The Set Bolus Amount block is the culmination of variables used to set the bolus to the best means of delivery based on all of the variables being polled. The device uses an algorithm to make determinations based upon the variables and operator settings.

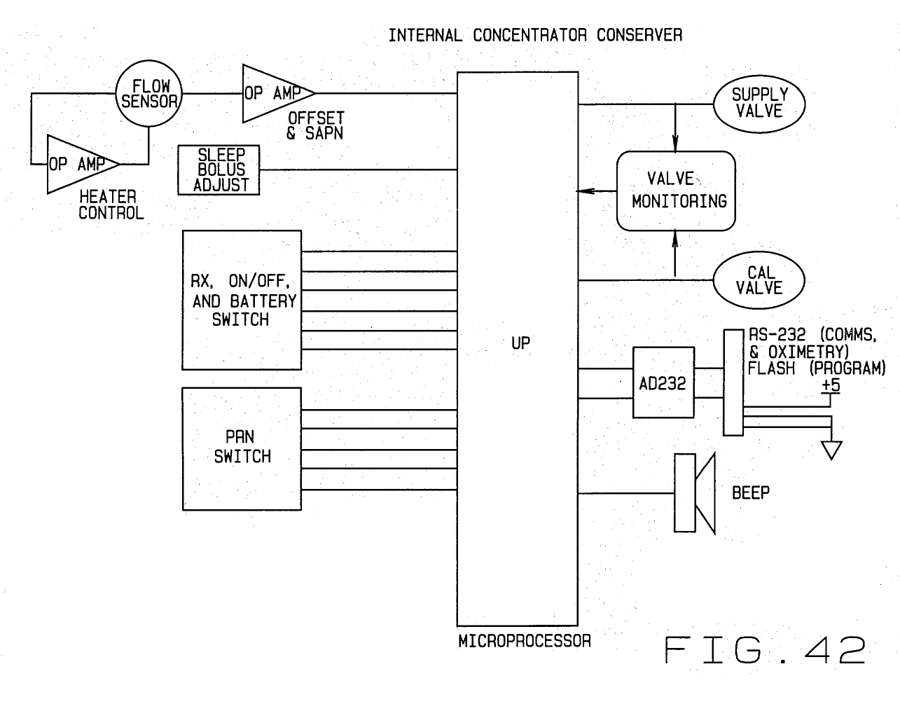

The Bolus Delivery block is the actual delivery of the desired bolus. The Rhythmic Artifact Rejection block provides for rejecting unwanted artifact. The flow diagram of FIG. 36 illustrates a preferred method to achieve rhythmic artifact rejection. As displayed in FIG. 35, activity generated artifact tends to be rhythmic in nature. A car driving down the road creates unwanted artifact and false deliveries for most conservers on the market today. The algorithm of the present invention monitors the expected breath rate and increases the setpoint level above null when artifact is present. If this does not remove the artifact effects, then the setpoint is increased once again. Periodically the algorithm goes to the default setpoint level to ensure an inhalation is not missed and to recheck for artifact and to once again fine-tune itself if artifact is present.

The Lockout block provides a time during a normal breathing cycle when signals from the flow sensor are disregarded. This time is often a fixed time after the delivery of a bolus for devices common in the art. The present invention may attempt to more precisely target each breathing cycle by creating a lockout time that is a percentage of the last breath time, for example 90% of the last breath time may be locked out of the current breath cycle.

The Reset Hysteresis block provides a method of monitoring signals from the flow sensor after the lockout time has passed and not allowing the device to enter Seek Mode until an inhalation is complete. It is common in the art for devices to double deliver, i.e., provide for the delivery of at least two boluses of oxygen, during long inhalations. An example of the double delivery is shown at FIG. 37, where the OM-400 is providing two deliveries. The double delivery wastes oxygen, reduces the capacity for ambulatory oxygen, and can cause some minor discomfort for patients. The preferred embodiment of the present invention provides for a two-step method wherein the flow sensor signal is monitored and must pass the test of receiving proper readings twice. An example of an applied two-step method may entail testing the flow sensor signal for proper readings, firstly at a level of 50 counts above Null, and then secondly at 25 counts above Null. This method insures that the inhalation is falling and that the patient is not in the middle of a long single inhalation.

The Periodic Calibration block periodically enables the Configure and Calibrate section discussed above. The periodic interval can be set to occur at a set time interval such as once an hour or may be activated whenever the device experiences changes in operation or environmental conditions. For example, a common temperature sensor could be input into the microprocessor to allow temperature monitoring and to trigger corrective action should it be required. Furthermore, the calibration sequence could be entered if the device senses a malfunction. FIG. 36 includes a section for periodic recalibration every 3600 seconds. This aspect of the present invention and the process elements of Calibration and Configuration block discussed above may be applied to a multitude of sensors for conserving devices, such as the aforementioned diaphragm sensor.

The Diagnostics, Program, and Event Recording block provides a method of gathering data including patient compliance data, usage, monitoring, recording, and transmitting the data such as the calculated oxygen contents, or measured contents remaining for both the device operator and for homecare providers. Such data may also include outlet gas temperature, outlet pressure monitoring, and the number of fills of therapeutic gas from a source to another gas delivery system. Data that will aid in diagnostics may also be provided in the present invention. Exemplary data that may be collected and used includes, but is not limited to the retention of BPM, activity, respiratory effort, respiratory rate, minute volume, heart rate, breathing waveforms, oxygen saturation, system backpressure including bolus backpressure, anticipation modes, apnea and hypopnea events and related information, snoring information, operator settings and the device settings both automatic and manual, bolus delivery profiles, calibration data and times, data concerning the sensing of inhalations, data on artifact rejection, lockout, hysteresis, external device operating data, and the failure of any section(s) to operate properly. The present invention may log all recordings and events to a related date/time stamp. The present invention may communicate any portion of the collected data to external devices for local or remote diagnostics. The present invention may also utilize telemetry in the communication of the data, and may additionally provide for control of a conserving device. The present invention may use either singly or a combination of real-time and recorded data. The data may then be used for control an external device or used to make control and operational decisions. The present invention may use a computer processor optionally including external memory. Additionally, the present invention may have the capability to receive programming and configuration data including actual programming data that will operate a conserving device. The present invention may also have the capability to transmit programming and configuration data including actual programming data that will operate an external device. When a battery is used by the present invention, the status of the battery may be monitored, displayed, recorded, and transmitted. The present invention may also provide audible alarms with distinct signals including display of any or all of the aforementioned data in this section. The present invention may also transmit alarm data for receipt by a suitable radio, modem, or optical device. In addition, the alarms may also be transmitted via non-auditory signals such as a vibration, puffs of a gas, or light.

The system default settings of the present invention may be restored manually or automatically by the control used in the conserver.

The present invention describes a use of an Alarm-through-cannula to provide alarm notification to the patient. Since the present invention enables the use of conserving technology remotely from the gas source, a patient may not be able to hear an alarm condition that is generated at the gas source or conserver. The present invention includes the means to transmit audio sounds into the device end of the patient outlet, through extension tubing and through the cannula to the patient's nares. The sound enters the patient's sinus area and is clearly discernable by patients with normal hearing. Additionally, the pulsing of gas is another means providing alarm notification from the device to the patient through the patient cannula.

The present invention provides for the optional use of a leak-through valve that provides an intended small leak through the valve. One of the advantages of the leak-through valve is that the flow-through sensor is flushed with oxygen or therapeutic gas. Additionally the leak-through valve provides a small positive flow to the patient's nares to prevent expired gases from entering the nasal cannula. It is also intended that the process of quickly firing a short burst or a group of bursts prior to the patient inhalations to clear the cannula of unwanted gases be part of the present invention.

Continuous flow provides a manual or automatic bypass of the supply valve for the purpose of selecting a continuous flow should a malfunction occur or for patient reassurance. To accomplish this task, a manual pneumatic switch may simply bypass the supply valve, or a valve disposed across the supply valve may be caused to flow should a malfunction occur. Selectable or proportional orifice means may be employed to allow control of flow to the patient in a continuous flow condition. Such orifice means may also be operatively connected to the RX and/or PRN selector switches that may be used in conserver mode.

Continuous Flow Interrupt provides a means of gathering data such as inhalation profile or BPM when a device operator has selected a continuous flow mode. This step acts to periodically stop the flow of gas to the patient and to gather flow data as described herein and then to resume normal continuous flow. In this embodiment of the present invention a dual lumen cannula may be used to continuously monitor the patient flow data and gas delivery in any mode of operation.

The Pressure Boost System provides a device and method for generating increased delivery pressures when for example, the oxygen product gas pressure is low and the length of patient cannula is long. The present invention provides for the option of boosting or intensifying gas pressure from one pressure to a higher delivery pressure. One example to accomplish this task is to feed product gas directly to a piston-based cylinder and to deliver the bolus directly from the cylinder to the patient. The cylinder may be pneumatically or electrically driven. Increased Delivery Pressure provides a means for creating a higher delivery pressure using a compressing means operatively connected to a reservoir. An example embodiment of the present invention describes an application to an existing LOX delivery system application where the outlet pressure may be too low to achieve the desired bolus at the patient end. Control of this feature of the present invention may be through the use of bolus pressure readings previously discussed.

Valve-at-Patient provides for displacing the supply valve to the patient end of the extension tubing or cannula. This system may use a single two-way valve that is powered by small power wires running from the conserving device through the cannula or along the cannula to the valve. Additionally, it is intended that the sensor means may be located between the valve and the patient's nares. An additional control wire may be run through the patient cannula or along said cannula. It is also intended that signals could be modulated along the power wires to lower the number of wires between the sensor and the conserving device at the oxygen or therapeutic gas source. Additionally, a three-way-valve-means may be used with a dual lumen cannula, or a dual sensing tube from the valve to the flow sensor located in the conserving device at the gas source. The Valve-at-Patient apparatus is particularly applicable to the situation when the source gas is at a low pressure. The cannula actually becomes a reservoir for the valve, allowing the delivery of a bolus that is not diminished by the length of cannula or extension tubing. Additionally, the valve means may be used as a coupler between the patient cannula and extension tubing. Additionally, it is intended that a Cal Valve as previously discussed may be located at the conserving device when the supply valve is remotely located at the patient side.

Oxygen Generating Device including Intelligent Conserving Method and Oxygen Cylinder Filling Method. The present invention may provide for an oxygen generating means, such as a PSA oxygen concentrator, to supply therapeutic oxygen to a demand conserving device. The conserver supplies oxygen to a patient at the relative beginning of an inspiration. When the conserver is not supplying oxygen to the patient, the oxygen is diverted through a valve means to a pressure intensifier or pump. This method of selectively filling a gas reservoir greatly speeds the fill time of said gas reservoir versus gas source devices that exist today. The intensifier supplies oxygen to a high-pressure oxygen cylinder and is capable of filling said cylinder to a predetermined high-pressure level for future ambulatory use by a patient. A flow control method is included within the scope of the invention to allow the device operator to select the equivalent flow delivery amount, or for continuous flow as follows. Additionally, an internal high-pressure cylinder or external high pressure cylinder feeding into the device is provided, allowing the use of a continuous oxygen delivery mode and an emergency back up supply of oxygen for continuous or demand mode delivery. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, it will be within the ability of one of ordinary skill in the art to make alterations or modifications to the present invention, such as through the substitution of equivalent materials or structural arrangements, or through the use of equivalent process steps, so as to be able to practice the present invention without departing from its spirit as reflected in the appended claims, the text and teaching of which are hereby incorporated by reference herein. It is the intention, therefore, to limit the invention only as indicated by the scope of the claims and equivalents thereof.

What is claimed is:

1. A therapeutic gas conserver and control device for a source of therapeutic gas comprised of:
a supply valve having a first side, a second side, and a supply side, wherein said supply side of said supply valve is connected to said source of therapeutic gas;
a flow-through sensor having a first side and a second side, wherein said second side of said sensor is connected to said first side of said supply valve;
a cal valve having a first side and a vent side, wherein said first side of said cal valve is connected to said first side of said flow-through sensor; and the said vent side is open to atmospheric air;
a patient outlet having a first side and a second side, wherein said first side of said patient outlet is connected to said second side of said supply valve;
at least one cannula having a first side and a second side, wherein said first side of said at least one cannula is connected to said second side of said patient outlet; and
a processor in electrical communication with said supply valve, said cal valve, and said flow-through sensor, wherein said processor provides a signal to control delivery of a portion of said source of therapeutic gas to a patient through said second side of said at least one cannula,
wherein said device delivers therapeutic gas to a patient.

2. The device in claim 1 wherein said at least one cannula is a dual lumen cannula.

3. The device in claim 1 further comprising:
an extension tube connected between said second side of said patient outlet and said first side of said at least one cannula.

4. The device of claim 1 further comprising:
at least one bacterial filter connected between said second side of said patient outlet and said first side of said at least one cannula.

5. The device of claim 1 further comprising:
an alarm in electrical contact with said processor.

6. The device of claim 5, wherein said alarm provides an auditory signal through said at least one cannula.

7. The device of claim 5 wherein said alarm provides a pulse of a gas signal through said at least one cannula.

8. The device of claim 1 further comprising:
a pulse oximetry measurement device in electrical communication with said processor.

9. The device of claim 1 further comprising:
a pressure transducer connected between said second side of said supply valve and said first side of said patient outlet, wherein said pressure transducer measures a pressure of delivery of said bolus of oxygen.

10. The device of claim 9 wherein said pressure transducer is in electrical communication with said processor.

11. The device of claim 1 further comprising:
a bypass, wherein said bypass provides a connection between said source of oxygen and said patient outlet.

12. The bypass of claim 11 wherein said bypass is in electrical communication with said processor.

13. The bypass of claim 12 wherein said bypass is controlled by said processor.

14. The device of claim 12 wherein said bypass is a manually set pneumatic switch.

15. The device of claim 12 wherein said bypass is a valve.

16. The device of claim 12 wherein said bypass is an orifice.

17. The device of claim 16 wherein said orifice is a selectable orifice.

18. The device of claim 16 wherein said orifice is a proportional orifice.

19. The device of claim 1 wherein said supply valve allows a desired quantity of said source of oxygen to continuously flow through said supply valve.

20. The device of claim-1 further comprising a supply compressor connected between said source of oxygen and said supply side of said supply valve, wherein said supply compressor raises the pressure of a supply of oxygen through said supply valve.

21. The supply compressor of claim 20 wherein said supply compressor is pneumatically powered.

22. The supply compressor of claim 20 wherein said supply compressor is electrically powered.

23. The supply compressor of claim 20 wherein said supply compressor is in electrical communication with said processor.

24. The supply compressor of claim 20 wherein said supply compressor is controlled by said processor.

25. The device of claim 1 further comprising a bypass compressor connected between said source of therapeutic gas and said first side of said patient outlet, wherein said bypass compressor raises the pressure of a supply of oxygen through said patient outlet.

26. The bypass compressor of claim 25 wherein said bypass compressor is pneumatically powered.

27. The bypass compressor of claim 25 wherein said bypass compressor is electrically powered.

28. The bypass compressor of claim 25 wherein said bypass compressor is in electrical communication with said processor.

29. The bypass compressor of claim 28 wherein said bypass compressor is controlled by said processor.

30. The device of claim 1 wherein said source of therapeutic gas is oxygen.

31. The device of claim 30 wherein said source of oxygen is an oxygen supply line from a centralized reservoir of oxygen.

32. The device of claim 30 wherein said source of oxygen is a tank of compressed oxygen.

33. The device of claim 30 wherein said source of oxygen is an oxygen generator.

34. The device of claim 33 wherein said oxygen generator is a ceramic oxygen generator.

35. The device of claim 1 whereas said at least one cannula is a pneumatic circuit terminated at a point of a patient inspiratory device.

36. The device in claim 1 further comprising:
a vent tube having a first end, a second end, and a tube volume, wherein said first end of said vent tube is open to atmospheric air and said second end of said vent tube is connected to said vent side of said cal valve;
wherein a volume of gas displaced during respiration of said patient is less than said vent tube volume.

37. The device in claim 1 further comprising:
a vent tube having a first end, a second end, and a tube volume, wherein said first end of said vent tube is connected to said first end of said cal valve and said second end of vent tube is connected to said first end of the flow through sensor;
wherein a volume of gas displaced during respiration of said patient is less than said vent tube volume.

38. The device in claim 1 whereas the processor is battery powered.

39. The device of claim 32 further comprising:
a backup supply of oxygen, wherein said backup supply of oxygen is connected to said supply side of said supply valve.

40. The device of claim 1 further comprising:
a backup supply of oxygen, wherein said backup supply of oxygen is connected to said first side of said patient outlet.

41. A therapeutic gas conserver and control device for a source of therapeutic gas comprised of:
A therapeutic gas supply system;
a flow-through sensor connected to said therapeutic gas supply system;
a vent system connected to said flow-through sensor; and
a processor in electrical communication with said therapeutic gas supply system, and said flow-through sensor, and said vent system.

42. The device of claim 41 wherein said vent system is comprised of:
a cal valve; and
a vent tube connected to said cal valve.

43. The device of claim 41 wherein said therapeutic gas supply system is comprised of:
a supply valve; and
a patient outlet connected to said supply valve.

44. The device of claim 43 wherein said therapeutic gas supply system is further comprised of:
a bypass connected between said source of therapeutic gas and said patient outlet.

45. The device of claim 44 wherein said bypass is in electrical communication with said processor.

46. The device of claim 44 wherein said therapeutic gas supply system is further comprised of:
at least one cannula connected to said patient outlet.

47. The device of claim 46 wherein said therapeutic gas supply system is further comprised of:
an extension tube connected between said patient outlet and said at least one cannula.

48. The device of claim 46 wherein said therapeutic gas supply system is further comprised of:
a pressure transducer connected between said supply valve and said at least one cannula, wherein said pressure transducer is in electrical communication with said processor.

49. The device of claim 46 wherein said therapeutic gas supply system is further comprised of:
a bacterial filter connected between said supply valve and said at least one cannula.

50. The device of claim 41 further comprised of:
an alarm in electrical contact with said processor.

51. The device of claim 41 further comprised of:
a pulse oximetry measurement device in electrical contact with said processor.

52. The device of claim 41 further comprised of:
a supply compressor connected between said source of therapeutic gas and said therapeutic gas supply system.

53. The device of claim 52 wherein said supply compressor is in electrical communication with said processor.

54. The device of claim 53 wherein said supply compressor is controlled by said processor.

55. The device of claim 41 further comprising:
a backup supply of therapeutic gas connected to said therapeutic gas supply system.

56. A method of conserving and controlling the delivery of a source of therapeutic gas to a patient, the steps comprised of:
providing a flow-through sensor in electrical communication with a processor;
providing a therapeutic gas supply system in electrical communication with said processor;
measuring at least one parameter of respiration of said patient;
providing an algorithm for said processor to evaluate said measured at least one parameter of respiration;
adjusting delivery of at least one bolus of therapeutic gas from said source of therapeutic gas to said patient in response to said evaluation of said at least one measured parameter of respiration.

57. The method of claim 56 wherein said at least one parameter of respiration is selected from the group consisting of respiratory effort, respiratory rate, activity, blood oxygen level, minute volume, heart rate, apnea, and hypopnea.

58. The method of claim 56 further comprising:
providing an algorithm for said processor to calibrate and configure use of said source of therapeutic gas, said flow-through sensor, and said therapeutic gas supply system.

59. The method of claim 58 whereas said calibration is in response to at least one environmental condition.

60. The method of claim 56 further comprising:
providing an algorithm for said processor to periodically calibrate use of said source of therapeutic gas, said flow-through sensor, and said therapeutic gas supply system.

61. The method of claim 56 further comprising:
automatically activating said measurement of at least one parameter of respiration from a signal by said flow-through sensor sent to said processor.

62. The method of claim 56 further comprising:
providing an algorithm for said processor to estimate at least one parameter of delivery of subsequent said at least one bolus of therapeutic gas from said source of therapeutic gas to said patient; and
delivering said at least one bolus of therapeutic gas according to said at least one estimated parameter of delivery.

63. The method of claim 62 wherein said parameter of delivery is selected from the group consisting of time, frequency, duration, volume, and pressure.

64. The method of claim 56 further comprising:
providing an algorithm for said processor to determine the occurrence of apnea events by evaluation of said measured at least one parameter of respiration.

65. The method of claim 64 further comprising:
communicating information of said apnea events determined by said processor to a receiving device.

66. The method of claim 64 further comprising:
communicating an alarm signal of said apnea events determined by said processor.

67. The method of claim 64 further comprising:
storing data of said apnea events determined by said processor.

68. The method of claim 56 further comprising:
providing an algorithm for said processor to evaluate and reject a rhythmic artifact.

69. The method of claim 56 further comprising:
providing an algorithm for said processor to provide a lockout time, whereby signals from said flow-through sensor are disregarded for a duration of time.

70. The method of claim 56 further comprising:
providing an algorithm for said processor to provide reset hysteresis.

71. The method of claim 70 whereas said lockout time is a percentage of the time between at least two previous patient inspirations.

72. The method in claim 56 wherein said therapeutic gas is pressurized atmospheric air.

73. The device of claim 41 wherein said therapeutic gas source is atmospheric air under pressure.

74. The device of claim 41 wherein said therapeutic gas source is oxygen under pressure.

75. A method of conserving and controlling the delivery of a source of therapeutic gas to a patient, the steps comprised of:
providing a diaphragm-based sensor in electrical communication with a processor;
providing a therapeutic gas supply system in electrical communication with said processor;
measuring at least one parameter of respiration of said patient;
providing an algorithm for said processor to evaluate said measured at least one parameter of respiration;
adjusting delivery of at least one bolus of therapeutic gas from said source of therapeutic gas to said patient in response to said evaluation of said at least one measured parameter of respiration.

76. The method of claim 75 wherein said at least one parameter of respiration is selected from the group consisting of respiratory effort, respiratory rate, activity, blood oxygen level, minute volume, heart rate, apnea, and hypopnea.

77. The method of claim 75 further comprising:
providing an algorithm for said processor to calibrate and configure use of said source of therapeutic gas, said diaphragm-based sensor, and said therapeutic gas supply system.

78. The method of claim 77 whereas said calibration is in response to at least one environmental condition.

79. The method of claim 75 further comprising:
providing an algorithm for said processor to periodically calibrate use of said source of therapeutic gas, said diaphragm-based sensor, and said therapeutic gas supply system.

80. The method of claim 75 further comprising:
automatically activating said measurement of at least one parameter of respiration from a signal by said diaphragm-based sensor sent to said processor.

81. The method of claim 75 further comprising:
providing an algorithm for said processor to estimate at least one parameter of delivery of subsequent said at least one bolus of therapeutic gas from said source of therapeutic gas to said patient; and
delivering said at least one bolus of therapeutic gas according to said at least one estimated parameter of delivery.

82. The method of claim 81 wherein said parameter of delivery is selected from the group consisting of time, frequency, duration, volume, and pressure.

83. The method of claim 75 further comprising:
providing an algorithm for said processor to determine the occurrence of apnea events by evaluation of said measured at least one parameter of respiration.

84. The method of claim 83 further comprising:
communicating information of said apnea events determined by said processor to a receiving device.

85. The method of claim 83 further comprising:
communicating an alarm signal of said apnea events determined by said processor.

86. The method of claim 83 further comprising:
storing data of said apnea events determined by said processor.

87. The method of claim 75 further comprising:
providing an algorithm for said processor to evaluate and reject a rhythmic artifact.

88. The method of claim 75 further comprising:
providing an algorithm for said processor to provide a lockout time, whereby signals from said flow-through sensor are disregarded for a duration of time.

89. The method of claim 75 further comprising:
providing an algorithm for said processor to provide reset hysteresis.

90. The method of claim 88 whereas said lockout time is a percentage of the time between at least two previous patient inspirations.

91. The method in claim 75 wherein said therapeutic gas is pressurized atmospheric air.

92. The device of claim 1 wherein said supply valve is remotely located near said patient and, said supply valve is in electrical communication with said processor, and said communication method between said supply valve and said processor is through electrical wires within said cannula.

93. The device of claim 1 wherein said flow-through sensor is remotely located near said patient.

94. The device of claim 93 wherein said electrical wires conduct modulated signals to said flow-through sensor and said supply valve along said electrical wires.

95. The device of claim 93 wherein the electrical wires are the power supply wires.
